(19) United States
(12) Patent Application Publication
(10) Pub. No.: US 2024/0115754 A1
(43) Pub. Date: Apr. 11, 2024

(54) ULTRA LIGHT BIOLOGICAL SATELLITE MASK REMOVABLE FROM AND/OR MATEABLE TO MECHANICAL, CHEMICAL, AND/OR NUCLEAR HOST MASK

(71) Applicant: Dynamics Inc., Cheswick, PA (US)

(72) Inventors: Jeffrey D Mullen, Glenshaw, PA (US); Keith Huthmacher, Pittsburgh, PA (US); David Malarik, Worthington, PA (US)

(21) Appl. No.: 18/484,415

(22) Filed: Oct. 10, 2023

Related U.S. Application Data
(60) Provisional application No. 63/414,894, filed on Oct. 10, 2022.

(51) Int. Cl. A61L 9/20

(52) U.S. Cl. CPC A61L 9/20; A61L 2209/12; A61L 2209/14; A61L 2209/15

(57) ABSTRACT

A full facial host mask is provided that may have a portion that may be removed and re-mated with the host mask. The removable portion may be, for example, a biological mask (e.g., a UV-C device that may include several UV-C light sources (e.g., UV-C LEDs) and such UV-C LEDs may have UV-C reflecting structures arranged to direct UV-Cina particular direction and at a particular size and shape). The UV-C generating device in the removable satellite mask ay be utilized in the breathing stream to provide a low-air resistance, long duration, high performance mask for biological (e.g., DNA-based, RNA-based, gram-positive bacteria, and gram-negative bacteria). The full facial mask may include mechanical filters (e.g., filters having 300 nm pores or smaller), chemical filters, and/or nuclear particulate filters. In doing so, a single mask may be issued to an entity (e.g., a warfighter) that can provide a full facial mask as well as partial face mask so various mission profiles may be achieved.

### CROSS-REFERENCE TO RELATED APPLICATION

This application claims the benefit of Provisional Patent Application No. 63/414,894, filed on Oct. 10, 2022, titled “ULTRA LIGHT BIOLOGICAL SATELLITE MASK REMOVABLE FROM AND/OR MATEABLE TO MECHANICAL, CHEMICAL, AND/OR NUCLEAR HOST MASK,” which is hereby incorporated by reference herein in its entirety.

### BACKGROUND OF THE INVENTION

This invention relates to sterilization.

### SUMMARY OF THE INVENTION

A UV-C generation device, such a device inside a face mask, is provided that includes multiple UV-C light emitting diodes (“LEDs”) positioned around a work area. For example, the multiple UV-C LEDs may be positioned around a cylinder. The cylinder may be, for example, comprised of a UV-C transparent material (e.g., a material with UV-C transparency greater than fifty percent (50%) such as, for example, quartz or UV-C transparent polymer. The LEDs may be located on a flexible printed circuit board. The flexible printed circuit board may be fabricated, for example, from a polyimide or FR4 and may be, for example between 2 thousandths of an inch and seven thousandths of an inch thick (e.g., between 2 and 4 thousandths of an inch thick such as between 2 and 2.5 thousandths of an inch thick). A working substance (e.g., a gas, a liquid, an air and liquid, a virus solution for inactivation for vaccine creation) may flow through the cylinder and the UV-C LEDs may interact with the working substance to, for example, sterilize the working substance. The UV-C LEDs may, for example, have a wavelength between 200 and 280 nanometers (e.g., between 220 and 280 nanometers or between 250 and 265 nanometers or between 255 and 260 nanometers such as 255, 260, or 265 nanometers).

Each UV-C LED may be independently controlled and regulated through control and regulation circuitry on the flexible printed circuit board or another device. Accordingly, the intensity of each UV-C LED as well as the turn-ON time and turn-OFF time of each UV-C LED may be independently controlled. A processor may be provided on the flexible circuit board or on another communicatively coupled device to control the operation of the UV-C LEDs.

The flexible printed circuit board may be, for example, wrapped around all of, or a portion of, the cylinder so that the UV-C LEDs may provide UV-C light into the cylinder through the cylinder wall. UVC-LEDs may be arranged in rows and columns. A UV-C flexible circuit when wrapped around a cylinder may, for example, have rows of three (3) UV-C LEDs in multiple columns (e.g., three columns, six columns, nine columns, twelve columns, more than twelve columns, or any number of columns). Accordingly, six columns of three UV-C LEDs would provide eighteen UV-C LEDs. The UV-C LEDs may be aligned in rows or staggered in rows around the cylinder. Persons skilled in the art will appreciate that the workspace may not be provide din a cylinder but in any shape that provides a workspace (e.g., inside a cube, rectangular, triangular, or any other type of housing).

UV-C reflective material may be provided on the flexible printed circuit board around the UVC-LEDs or selectively provided, around the UV-C LEDs placement so as to not generally impede UV-C emanating from the UV-C LEDs, on the interior surface or exterior surface of the cylindrical housing. Such a UV-C reflective material may include, for example, aluminum.

One or more heat sinks may be provided around the UV-C LEDs in order to capture and expel heat from UV-C LEDs away from those UV-C LEDs. A battery and/or wall plug and/or battery and wall-plug may be utilized to charge, for example, one or more rechargeable batteries located inside a housing that includes the working space.

Manual inputs may be operable to receive manual input from outside of a housing that may include the working area (e.g., a UV-C transparent cylinder) or be placed within the proximity of a working area. Temperature, humidity, and flow rate may be added and utilized to, for example, control the intensity of one or more of the UV-C LEDs so that, for example, the intensity may be changed for different temperatures, flows, and/or humidity.

Persons skilled in the art will appreciate that other types of Ultraviolet LEDs, or other light sources, may be provided on an LED array such as UV-B and UV-A LEDs. Similarly, additional wavelengths of light may be provided in LEDs, or other types of light sources. A spectrometer, or other device, may be included to determine the type of material in the working space and may activate different LEDs or different types of LEDs (e.g., based on the detected material(s)). Similarly, different UV-C LEDs, or non-LED UV-C sources, may provide different wavelengths and different modes may be provided to control the UV-C LEDs so a subset of the UV-C LEDs may provide a particular nanometer wavelength (e.g., 255 to 265 nanometers) and other UV-C LEDs may provide another particular nanometer wavelength (e.g., 270 to 280 nanometers).

A flexible circuit board does not have to be rolled, for example, for the flexible circuit board to sterilize a working surface. A device may have a generally flat flexible circuit board at a perimeter separated from a surface that has contaminant (e.g., virus and/or bacteria) that requires sterilization) The housing may have a handle (e.g., a removable handle) so that the UV-C sterilization device can be provided as want for moving over, and sterilizing, a surface.

The housing may include multiple mateable ports for handles such that, for example, one handle may be inserted into one mateable port to provide a sanitizing and a larger handle may be inserted into a different mateable port to provide a sanitizing mop/broom. Such a UV-C sanitizing device may be wall mounted such that, for example, someone can place their hands in a working space and have the hands sterilized. The device may operate on two modes—human mode and non-human mode. The device can prompt this to the user for the mode, wait for the user to activate the mode, or autonomously activate the mode.

The flexible circuit board with multiple UV-C LEDs may be articulated via motors and/or other controls so that different areas that, for example, include UV-C LEDs may be moved away from each other or to each other or moved closer to, or further away from, the other LED's.

Persons skilled in the art will appreciate that a fixed distance surface cleaner may be utilized. A fixed distance surface cleaner may be, for example, permanently attached (e.g., bolted and/or screwed) to a surface (e.g., a counter-top) so that objects may be passed in front of UV-C generating portion(s) to sterilize the objects. For example, a UV-C surface sanitizer may be provided on a countertop next to a point-of-sale register. A customer may pass a credit card and or a currency bill and/or a coil under a UV-C sanitization device to sanitize a device. A UV-C generating device may be embedded in the countertop or placed in the countertop and may face upwards so an object provided over it may be sanitized on the surface(s) facing the UV-C generation. UV-C generation units may provide a particular amount of UV-C light at a particular point and may be controlled, over time, to provide that amount of UV-C light at that particular point. Accordingly, for example, UV-C light may be provided at an amount that sterilizes at a particular distance (e.g., under 5 millimeters from the surface of a counter) but not at a further point (e.g., beyond 5 millimeters) from the surface of a counter. UV-C generators may be provided over and/or under a conveyer (e.g., a gapped and/or conveyer with UV-C transparent material).

A UV-C air sterilization device is provided in which a fan (e.g., axial fan and/or centrifugal fan) pushes and/or pulls air through a working area into which UV-C is applied. The air may then be directed over the UV-C sources of light so that the sterilized air is also used to remove heat from the UV-C sources. The circulated air that has been sanitized and utilized to remove heat from the sanitization device may then be, for example, expelled from the device. In doing so, the device may move sanitized air from the device without moving non-sanitized air from the device.

An air sanitization device may also apply other types of light such as UV-A and/or UV-Blight in addition to, or in place of, UV-C light. A fan may have several speeds such that different efficacies of sterilization may be provided and/or different air speeds may be provided.

One or more fixed and/or removable mechanical particulate filters may be provided (e.g., before the working area of the UV-C sanitization device). In doing so, particulates may be kept away from A UV-C working area of the device.

One or more (e.g., several) speed settings may be provided to circulate air through a UV-C working area. Such various speeds may, for example, provide different impact rates (e.g., inactivation rates) of various air-born contaminants (e.g., virus) and may provide different speeds at sanitizing air.

An autonomous cleaning operation may be provided by a UV-C sanitization device that may clean a UV-C generating device. For example, an air sterilization device may utilize one or more fans to move air through a UV-C working area at a maximum speed during operation. However, during cleaning, the one or more fans may move the air through the UV-C working area at a faster rate and such a faster rate may be constant for a period of time or may include several pulses of air. A cleaning substance may also be released to be moved through the working area during an autonomous leaning operation. A portion of a UV-C air sterilization device may be accessible to a user so that the user may, for example, access a UV-C working area of a UV-C air sanitization device for cleaning. Cleaning objects (e.g., a brush that can fit into the working area of a UV-C sanitization device, cloth, and/or other object may be provided in a sealed box with the UV-C air sanitization device for consumer sale). A UV-C sanitization device may have an indicator (e.g., verbal and/or audible) to provide a notification to a user that a user-driven and/or user-assisted cleaning process is desired. A housing of a UV-C sanitization device may include, for example, a mating structure such that a cleaning object may be mated to the UV-C sanitization device.

One or more light sources (e.g., visible light sources) may be placed in one or more working areas of a UV (e.g., UV-A, UV-B, and/or UV-C) air sanitization device and one or more sensors that can detect the light provided from those light sources may be placed in the working channel or areas where light from the light sources may reach. Persons skilled in the art will appreciate that different intensities of light sensed may, for example, be indicative of different amounts of residue (e.g., dirt and/or dust) that may have gathered on the surfaces of a UV-C working area as different amounts of residue may decrease, for example, the reflectivity of the surfaces with the reside. Persons skilled in the art will appreciate that materials that are transparent to particular wavelengths may be utilized in a UV-C working area. Light (e.g., visible and/or non-visible light) may be provided through these transparent materials and sensors may be utilized to determine any residue on such transparent materials. Accordingly, light sources (e.g., visible light and/or non-visible light sources) may be utilized with sensors to determine the state of cleanliness of UV-C working surfaces by detecting different amounts of residue. Additionally, for example, UV-C sensors may be utilized to determine the amount of UV-C light in particular areas to determine, for example, how much reflectivity and/or transparency has been degraded from residue over reflective and/or transparent materials in and/or around a UV-C working area, respectively. Residue may be, for example, determined by direct sensing means such as for example a camera that takes a picture and analyzes the picture.

A reflective perimeter may be placed around a UV-C light source such that, for example, UV-C light is directed in a particular direction. Additionally, for example, UV-C reflective materials may be utilized to improve UV-C mating between a UV-C LED and a UV-C transport medium (e.g., a UV-C fiber optic).

UV-C may be utilized to inactivate amounts of a virus (e.g., SARS-CoV-2) in a substance, such as the air, order to create a vaccination such as an aerosolized vaccination. Inactivated virus may then be breathed in to have a vaccination impact. Such an aerosolized vaccination, or another form (e.g., liquid) vaccination may be provided by an inactivation fan that inactivates air or in a ventilator, or other medical device, as an air inactivation device. In an example of ventilator, or other device, fans may not be provided to move air through an inactivation working area as the ventilator, or other device, may utilize move air, or another substance (e.g., liquid), through the inactivation working area. UV inactivation of virus to create vaccines may be performed, for example, with UV-C. Multiple strains of virus (e.g., strains from different claves of virus) may be inactivated and combined in order to form a super vaccination across one, two, or more than two virus, strains of virus from the same clave, strains of virus strains of a virus from different claves. For example, a multi-strain vaccination may include strains of a virus from at least 3 or at least 5 different claves. Accordingly, for example, a multi-clave vaccination may be provided by inactivating with UV-Cone or more virus strains from multiple or several claves of SARS-CoV-2 and combining the inactivates virus strains in a single vaccine for administration to a human being. Persons skilled in the art will appreciate that the amount of different strains of a virus may be the same. A vaccination may have any number of inactivated virus such as, for example, one million, ten million one hundred million, one billion, or more than one billion virus and may have one inactivated strain, more than one inactivated strain, and the inactivated strains may be provided in equal proportions or different proportions.

One or more UV-C air sterilization devices may be, for example, placed in an air duct (e.g., 24 inch by 24 inch, 36 inch by 36 inch, 48 inch by 48 inch, circular air duct, and/or rectangular air duct) One or more UV-C air sanitization devices may be placed after an air register bringing air into an air duct and/or room or before an air register bringing out of an air duct and/or room. Such devices may be provide on a structure that forces all, or most, of the air to go through the UV-C air sanitization devices. Each air sanitization device may have, for example, one or more fans (e.g., two fans where each fan includes two counter-rotating blades). The structure may be expandable and collapsible so that the air sanitization device may be utilized in different size and/or shape air ducts. One or more controllers may be on the structure and/or one or more of the UV-C air sanitization devices that may control all of the devices (e.g., control which fans are ON/OFF and the speeds of each fans) and may receive information from the devices (e.g., if a device needs servicing such as UV-C LEDs need to be replaced to maintain a particular efficacy). Persons skilled in the art will appreciate that one or more redundant air sanitization devices may be included such that one or more of the air sanitization devices loose efficacy (e.g., UV-C LEDs fall below a performance threshold so the UV-C air sanitization devices falls below a performance threshold) redundant UV-C air sanitization devices may be turned ON. Alternatively, for example, all UV-C sterilization devices may be ON and the speed of fans (if included in an air sanitization device) may be adjusted based on the number of UV-C air sanitization devices in an array and the current operating efficacy of the array. Sensors may be utilized in the UV-C generating devices to determine the amount of UV-C being generated (e.g., by detecting UV-C light or another light emitted such as visible light, UV-B light, and/or UV-A light).

Persons skilled in the art will appreciate that UV-C generating devices may operate at different efficacies over time under different types of operating regimes as, for example, intensity of UV-C light sources decay (e.g., intensity of UV-C light emitting diodes degrade). For example, UV-C LEDs may be operated at high current and may decay faster in some instances than if those UV-C LEDs were operated at a lower current. As per another example, UV-C LEDs at a particular current may degrade different based on, for example, how an LED is pulsed. For example, a UV-C LED that is pulsed so it is ON more in a particular amount of time than a pulse regime where it is ON less in that particular amount of time may degrade faster. Program logic, such as program logic stored in a memory and run by a processing circuit (e.g., a processor) may, for example, estimate the intensity of UV-C light for one or more UV-C light sources (e.g., UV-C LEDs) over time. As modes are changed (e.g., intensity is turned down autonomously or manually), the estimation may be updated using stored information indicative of decay curves associated with those various modes or through other estimation processes (e.g., utilizing light sensors to determine the amount of light being produced by one or more light sources). One or more notification structures (e.g., visible spectrum LEDs, displays, speakers, and/or tactile generators) may provide human perceivable notifications periodically, upon request (e.g., manual request), or at particular events (e.g., each time a device is turned ON or a mode is changed). For example, three visible spectrum LEDs may be provided. Each visible spectrum LED may be associated with a different operational mode. Each operational mode may be associated with a different fan speed. Accordingly, for example, a consumer may select (e.g., via a mobile application on a mobile phone, a website on a device such as a laptop, or using an interface such as a toggle button on the device) a fan speed and a light corresponding to that van speed may provide an indication the associated mode has been selected. Accordingly, a fan may have, for example, a “low” airspeed, a “medium” airspeed, and a “high” airspeed. Airspeeds for a device may be, for example, 100 liters per minute (“LPM”), 200 LPM, and 400 LPM. For example, airspeeds may be different by at least 100 LPM (e.g., 100 LPM, 200 LPM, and 300 LPM). Alternatively, for example, airspeeds may be different by at least 500 LPM (e.g., 500 LPM, 1,000 LPM, and 1,500 LPM). One or more manual interfaces (e.g., one or more buttons or touch interfaces) may be provided (e.g., a touch-sensitive display may be provided). Such manual interfaces may select any number of modes (e.g., any number of fan speeds). A digital number may be provided associated with a fan speed on a display, for example, and a manual interface (which may be the display itself) may be utilized to increase and/or decrease the fan speed by the manually controllable increments of the device (e.g., 1 LPM or 1 cubic feet per minute CFM). A consumer may change between different metric systems (e.g., LPM or CFM) on a display in order to personalize the device to a desired metric system. Similarly, for example, a language (e.g., English, Japanese, etc.) may be selected and the system may provide notifications and other display screens in the desired language.

Persons skilled in the art will appreciate that devices may monitor the intensity, and other attributes, of light sources (e.g., UV-C LEDs) and utilize this information to estimate inactivation rates at a particular period of time/operation for a particular pathogen (e.g., a particular virus, spore, bacteria, etc.) and/or estimate inactivation rates based on any attribute (e.g., the time the device is ON in a particular mode). Accordingly, for example, a device may provide notifications for different inactivation thresholds. For example, a device may have an inactivation range of 90-99% for a virus (e.g., SARS-CoV-2) during a first efficacy period, an inactivation range of 75%-89.9 percent during a second efficacy period, an inactivation range of 50% to 74.9$ during a third efficacy period, and an inactivation range of 1 to 49.9% during a fourth efficacy period. Different modes (e.g., different speeds such as 100 LPM, 200 LPM, and 400 LPM) may have different times under different efficacy periods as the different modes may utilize different intensities of lights to achieve those efficacy periods (e.g., as slower moving air may utilize less UV-C light to inactivate at a particular efficacy and, as a result, utilize less energy and provide less degradation in the light source). Light sources may be operated in different wants for different modes and/or during different efficacy ranges. For example, one or more light-emitting diodes may be turned ON (e.g., pulsed at a particular rate) and the diodes may degrade over time and the efficacy of the one or more light-emitting diodes may decrease with time. As per another example, a current may be selected at a point in the efficacy range and as the light emitting diode degrades the current may be increased so that the degradation is countered. Doing so, for example, may extend the amount of time a device can provide efficacy at a particular efficacy level. Multiple operation regimes for one or more LEDs may be provided in an efficacy range. For example, one or more UV-C LEDs in a range of efficacy of 1% to 49.9% may first operate to sustain efficacy at 25% (e.g., by providing a current to provide 25% and then increasing current as the UV-C degrades) and then sustaining efficacy at 15% and then, after that is complete, running the UV-C LEDs to a lower amount (e.g., 1%). At the end of an efficacy range, the device may provide a fault code to the consumer (e.g., by flashing one or more visible spectrum LEDs in a sequence such as a countdown sequence and then showing a fault code) so the consumer is made aware that one or more UV-C LEDs should be changed (e.g., either via a consumer or via a third party). A communications antenna may be provided in the device and the device may communicate directly to a third-party light source provider or to a device of the user (e.g., a mobile phone) to notify the consumer of the need for a light-source change. A consumer may control any aspect of a UV-C inactivation device on any other device via wired or wireless communications between that device and the UV-C inactivation device (e.g., through one or more intermediary devices). Whenever a device is turned ON, changed to a new mode, or any other event, a user notification may be provided to indicate the efficacy mode of the device at that particular period of time. For example, two, three, or more than three LEDs may be utilized for different fan speeds. When a fan speed is entered a visible spectrum LED associated with that fan speed may not blink if it is in a first efficacy level, may blink twice if it is in a second efficacy level (e.g., and then the LED may stay ON), may be three times if it is in a third efficacy level, may blink four times if it is in a fourth efficacy level, and so on for any number of efficacy levels. Notifications may be sent to various devices (e.g., mobile phones associated with one or more users) as a result of the change in efficacy level (e.g., for a particular mode such as a particular fan speed). Persons skilled in the art will appreciate that a device, for example, may have a mode of operation, for example, having at least four efficacy range levels and may have at least, for example, 750 hours of operation in each of those efficacy range levels (e.g., at humidity greater than 50%). An efficacy range level may have over, for example, at least 1,000 hours of operation or, for example, at least 2,000 hours of operation.

Persons skilled in the art will appreciate that a device may be controlled not by fan speed but, for example, an efficacy (e.g., or both). For example, a user may select different efficacy ranges and the device may, if the efficacy ranges are available, drive the UV-C light sources to provide the desired efficacy. If the efficacy is no longer available, the user may receive a notification that the efficacy is no longer available (e.g., via visual indications such as visible LED indications and/or display indications). A device may include interfaces (e.g., a touch screen display) to provide controls of the device for any type of operation such as, for example, to provide a time extension operation (e.g., sustain light sources at a particular point, such as a lower point, of an efficacy level) or to maximize efficacy for a particular time (e.g., provide the most or a relatively higher current point through a light source for a particular time). In doing so, the lifetime of a device may be extended and the inactivation rate of a device may be increased depending on a particular application and/or situation for a particular period of time or environment. Persons skilled in the art will appreciate that a humidity sensor may be provided and readings from such a humidity sensor may be utilized to impact efficacy ranges at a particular time or utilized to conserve power (e.g., lowering current during parts of relatively lower humidity compared to a higher current during parts of relatively higher humidity).

Sound suppression structures and chambers may be added to a device in order to reduce the sound that can reach a user. Such sound suppressors may be utilized to reduce sound around a particular part or in a particular direction of a device. For example, a sound suppressor chamber may be provided before the inlet to air fans pulling air into a working area by providing a mechanical sound barrier (e.g., plastic and/or metal) in line with the fans at the inlet and having air move into a chamber and around the sound barrier so that sound is contained in the chamber. The chamber may be filled with soundproofing material (e.g., soundproofing foam) and may be more than two inches in length at its largest length point, two inches in width at its largest width point, and more than two inches deep at its largest depth point (e.g., more than two inches in height such as three inches in height or more than 3 inches in height). Soundproofing coatings may be provided on any surface. Furthermore, fans may be isolated in harnesses or via a mechanical absorbing structure such as a rubber so that vibrations in the fans are reduced in travel to other structures of the inactivation device.

Inactivated air may be pushed (e.g., or pulled) from an air inactivation device through any structure to, for example, exit the device. Accordingly, one or more fans may push air through an area in the shape of a circle, ellipse, rectangle, or annulus. A cone may be placed in the center of the annulus to assist air existing the annulus. The cone may be as long as, for example, at least half the diameter or at least the diameter of the inner wall of the annulus. The end of the cone may be pointed or rounded. The curvature of the cone may be constant or may change down the length of the cone. The beginning of portion of the cone may be a cylinder and may be form an interior wall, for example, to the annulus.

Different annulus structures may be placed in the middle of the annulus to provide a pathway and different resistances to output air. Such annulus structures may be removable and the device may recognize different structures. Such structures may be recognized, for example, by, for example, the mechanical, electrical, or other detection (e.g., optical) of different types of mating structures that are unique to each mated structure. Similarly, different types of other structures may be detected such as, for example, output funnels that go around the exterior of the output annulus to direct air. Persons skilled in the art will appreciate that different structure about the input and/or output of a fan may, for example, increase and/or decrease air pressure at the input and/or output of the fan.

Detection of additional structures, or detection of a change in airflow through an air inactivation device, can be utilized to, for example, autonomously change the airspeed to a pre-determined airspeed. Accordingly, for example, the addition or removal of a structure that impacts airflow may be sensed by sensing a change in airflow and fan speed, for example, may be changed to counteract. Accordingly, for example, a device that is operating at 100 liters per minute that has a concentration funnel or annulus cone added to the output and that may reduce the airflow may be sensed and fans speeds may be increased to return the device to, for example, 100 liters per minute. A device may have any number of settings such as, for example, any number of speed settings such as three speed settings (e.g., 100, 200 and 300 LPM or 100, 200, 400 LPM). A speed setting may be at least twice the speed of a particular speed setting and yet another speed setting may be at least three times the speed of a particular speed setting.

Accordingly, for example, one or more airflow sensor may be provided in the device. A setting may cause the unit to change fan speeds to achieve the setting. If the setting cannot be achieved, for example, a fan speed that provides the closest potential air speed to the setting may be provided. The device may look at the air speed setting and, if the air speed changes, the device may adjust the fans to re-achieve the desired setting. A manual input may be provided to provide the setting. Alternatively, for example, the device may have a particular airflow setting the device may be attempting to achieve.

Multiple air inactivation devices, or other types of inactivation devices, may be utilized collectively to inactivate an area of contaminants (e.g., virus) or to provide an inactivation strategy for an area, object (e.g., person), or objects (e.g., persons). In this manner, for example, inactivation devices may communicate data between each other and between, for example, a remote system such as a remote controller system. Accordingly, a number of air inactivation devices may be provided on one-axis, two-axis, or any number of axis oscillators and the fans may move in a coordinated fashion to achieve a strategy. A strategy may be to inactivate air in a room, to track and follow one or more objects and provide clean air to those objects (e.g., persons), or any other strategy. Sensors (e.g., vision systems) may be placed around a room or may be moved (e.g., via one or more vehicles such as wheeled vehicles). Inactivation devices may scan an environment when the fans are placed in an environment, engage in communication with nearby inactivation units, determine a strategy based on the capabilities of the inactivation units collectively working together, determine additional environmental conditions such as, for example, altitude, temperature, humidity, and execute a strategy together based on the information collected.

A working area for UV-C air inactivation may have, for example, one or more air inlets and one or more air outlets. UV-C reflective objects may be placed in the inlets and/or outlets such that light is reflected back into the working area but air is permitted to flow past and/or through the objects.

A facemask may be provided with a satellite mask that is removable from the facemask to provide, for example, a partial facemask. In doing so, a user (e.g., a warfighter) may be provided with one facemask for a particular mission profile (e.g., mechanical, chemical, radiological) and a smaller facemask may be detached for a different mission profile (e.g., biological). In doing so, the user may have the ability to utilize a full facemask when desired and a lightweight partial facemask when desired.

The full facemask may not, for example, be able to operate as a full facemask when the satellite mask is removed. A closure portion may be utilized when the satellite mask is removed to close the portion left void when the satellite mask is removed so that two functioning facemasks may be provided. The full facemask and the satellite facemask may mechanically couple through a mechanical attachment and re-attachment structure. A closure portion may attach to such a structure to, for example, close the full facemask so the full facemask can be utilized. Additionally, for example, peripherals may be added to the full facemask using this attachment and re-attachment structure such as, for example, sound amplification modules (e.g., to amplify the voice of a user), additional filters, or any other peripheral. Persons skilled in the art will appreciate that it may be desirous to remove a satellite mask if, for example, a mission profile is provided that the satellite mask decreases the efficacy of the mask when mated. In such an instance, for example, the satellite mask can be removed and a closure portion utilized to close, if applicable, any void(s) left by the removed satellite mask.

The satellite mask may include a UV-C inactivation chamber and may be, for example, battery operated. In doing so, for example, a low air resistance, high performance, pathogen inactivation mask may be provided that may utilized, for example, to inactivate biological pathogens. Affixation straps may be removable from the full facial mask and utilized with the satellite mask so that the same affixation straps may be utilized (e.g., to affix the mask to a user's head). One or more fans may be provided in the satellite mask to, for example, provide air cooling to UV-C sources providing energy into a working area. Heat sinks and/or heatsink fins may be utilized to remove heat from UV-C sources and air from one or more fans (e.g., two fans or more than two fans) may be utilized to move air across the heat sinks and/or heat sink fins). One or more air flow detectors may be utilized to determine the direction and magnitude of air flow. In doing so, for example, energy from UV-C sources may be increased (e.g., moved to a maximum permitted energy) when air is inhaled and decreased when air is exhaled (e.g., turned OFF). Persons skilled in the art will appreciate that UV-C sensors may be utilized to determine the amount of energy produced at a particular time by one or more UV-C sources and current to the UV-C sources may be increased based, for example, on the magnitude of air flow. Accordingly, a UV-C inactivation system may generate more energy when a user is breathing heavily (e.g., running) than when a user is breathing less heavily (e.g., walking) and generate even less energy when a user is breathing even less heavily (e.g., sleeping). Additionally, such a flow sensor may determine, for example, abrupt changes in airflow (e.g., coughing) and may change the inactivation rates by increasing energy and to a pre-determined threshold, which may be sensed and confirmed by one or more UV-C sensors, to provide a particular (e.g., pre-determined) inactivation rate (e.g., for a particular type of pathogen such as an RNA-based virus). Person skilled in the art will appreciate that inactivation exhaled pathogen may increase the safety of those in the proximity of a user without a mask as well as decrease the probability that an exhaled pathogen may be later inhaled and not stopped by another mask (e.g., a mechanical particular mask).

Persons skilled in the art will appreciate that one or more visual indicators (e.g., LED(s) and/or display(s)) may provide information to a user such as, for example, time until UV-C sources may need replenishing to keep a particular performance, the inactivation rates achieved for particular types of pathogens based on particular operating modes, as well feedback on a user's breathing patterns.

Fans in a satellite mask may be utilized, for example, to provide breathing assistance for the satellite mask and/or a full-face mask when the satellite mask is mated to the full facial mask. For example, the satellite mask may be mateable to additional filters such as mechanical, chemical, and/or nuclear particulate. As air resistance is added to filters, air from the fans may be utilized to move air in and out of such filters in order to decrease the perceived air resistance by the user. As such, assisted breathing may be provided.

Additional sensors may be provided in a primary mask (which may not be a full facial mask) or secondary 9 e.g., a satellite mask) such as, for example, humidity sensors, human pulse sensors, temperature sensors, or any type of sensor and information from such sensors may be utilized, for example, as part of a determination to change a mode of operation of one or more components of a mask (e.g., a UV-C inactivation structure of a mask).

A satellite mask may have mateable structures for a primary mask. Such mateable structures may be utilized to let a user change facial seal types on a mask such as change between a foam seal, air pillow seal, gel seal, rubber seal, etc. Air flow sensors, or other sensors, may determine, for example, that a seal effectiveness is decreased and the mask may change its mode of operation to assist (e.g., increase energy by UV-C devices and/or create an assisted breathing by trying to move air into the breathable proximity of a user so that non-inactivated air breaching the decreased integrity seal does not reach a breathing area (e.g., the mask may create an air mask around the user's breathing zone).

When a satellite mask is mated to a primary mask, the satellite mask may provide filtration for a particular type of mission (e.g., biological) while the primary mask may provide additional filtration for additional types of mission (e.g., mechanical particulate, chemical, and/or nuclear filament).

### BRIEF DESCRIPTION OF THE DRAWINGS

The principles and advantages of the present invention can be more clearly understood from the following detailed description considered in conjunction with the following drawings, in which the same reference numerals denote the same structural elements throughout, and in which:

FIG. 1 are illustrations of UV-C devices constructed in accordance with the principles of the present invention;

FIG. 2 are illustrations of UV-C devices constructed in accordance with the principles of the present invention;

FIG. 3 are illustrations of UV-C devices constructed in accordance with the principles of the present invention;

FIG. 4 are illustrations of UV-C devices constructed in accordance with the principles of the present invention;

FIG. 5 are illustrations of flow charts constructed in accordance with the principles of the present invention;

FIG. 6 is an illustration of UV-C device constructed in accordance with the principles of the present invention;

FIG. 7 are illustrations of flow charts constructed in accordance with the principles of the present invention;

FIG. 8 are illustrations of UV-C devices constructed in accordance with the principles of the present invention; and

FIG. 9 are illustrations of UV-C devices constructed in accordance with the principles of the present invention.

### DETAILED DESCRIPTION OF THE INVENTION

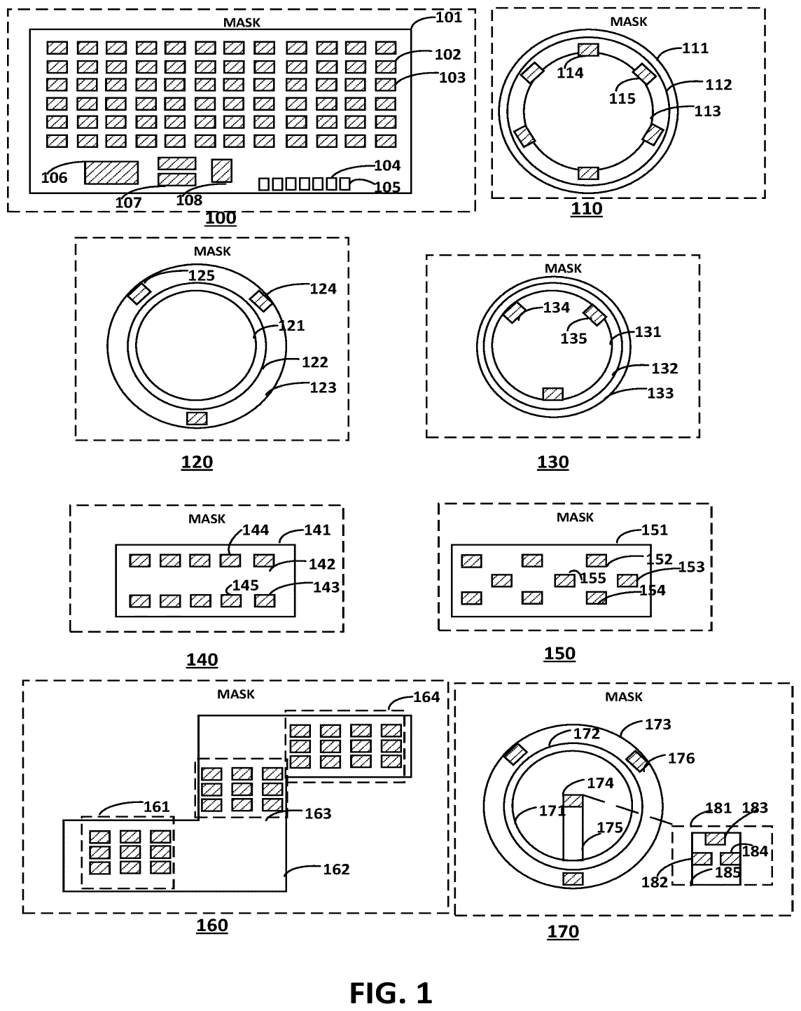

FIG. 1 shows device 100 that may be a mask (e.g., a primary mask, satellite mask, and/or multiple mateable masks) and may include any number of ultraviolet C (UV-C) light sources such as UV-C light emitting diodes 102 and 103. UV-C sources may have a wavelength between approximately 200 nanometers and 280 nanometers. Processor 106 and additional circuitry 107 may be included on circuit board 101 in additional to input/output ports 104 and 105.

Printed circuit board 101 may be, for example, a non-flexible or a flexible printed circuit board. Input/output ports 104 and 105 may be, for example, contacts to couple to another circuit board or an external device. Processor 106 may, for example, control UV-C LEDs 102 and 103 using firmware that is downloaded into processor 106 or provided in a memory of processor 106 before or after placement on circuit board 101. Persons skilled in the art will appreciate that printed circuit board 101 may be multiple printed circuit boards that are communicatively coupled together to form a multiple circuit board device. Different circuit boards of a multiple circuit board device may be provided in a single housing or in different housings.

Firmware updates may be downloaded through input/output ports 104 and 105. Any number of input/output ports may be provided and different protocols may be utilized for different ports. Additionally, blue-tooth (e.g., BLE), contactless (e.g., RFID), telecommunications (e.g., cellular such as 4G or 5G cellular), infrared, or other wireless communication structures may be provided such as wireless communication chips, circuitry, protocols, and ports may be provided. Wireless power generation may be provided and may be utilized by power circuitry to change a battery coupled to printed circuit board 101 (e.g., through battery contact pads on circuit board 101).

Printed circuit board 101 may be a flexible polyimide or flexible Fr$. Persons skilled in the art will appreciate that such a flexible printed circuit board may be, for example between two thousandths of an inch and seven (7) thousands of an inch in thickness (e.g., between two thousandths of an inch and three thousands of an inch in thickness). Silicon chips may be grinded and polished before placement on printed circuit board 101 to between, for example, five thousandths and ten thousandths of an inch in thickness). Such chips may be mounted on printed circuit board 1010 via a flip-on-flex structure or via a wire-bonded structure. A wire-bonded structure may be for example a low-provide wire-bonded structure with wire-bonds that are placed with less than a five thousandths of an inch profile above the silicon chip and encapsulant that is less than three thousandths of an inch above each wire-bond. The entire thickness from the bottom of flexible circuit board to the top of an encapsulant of a chip may be, for example under fourteen thousandths of an inch thick (e.g., under twelve thousandths of an inch thick). For example, the thickness from the bottom of circuit board 101 to the top of the encapsulant may be between ten and sixteen thousandths of an inch thick (e.g., between twelve and fourteen thousandths of an inch thick). Wire-bonds may be for example, gold wire-bonds or aluminum wire-bonds. A low-profile encapsulant may be provided that utilizes at least two separate encapsulate provisioning steps in order to provide the low-profile encapsulant.

Processor 106 may be one or more processors and may be provided between, for example, twenty megahertz and five gigahertz. Persons skilled in the art will appreciate that faster processors may provide faster control of UV-C LEDs 102 and 103. Faster control of UV-C LEDs may provide shorter ON times which may provide the ability to damage and sterilize certain elements (e.g., virus) without damaging and sterilizing other elements (e.g., living tissue and cells). Processor 106 may, for example, provide ON times for UV-C LEDs 102 and 103 less than, for example, 100 nanoseconds, less than 10 nanoseconds, less than 1 nanosecond. For example, Processor 106 may turn ON UV-C LEDs 102 and 103 between approximately 1 and 100 nanoseconds (e.g., between 20 and 60 nanoseconds or between 30 and 50 nanoseconds). High speed control circuitry may also be provided in order to control UV-C LEDS 102 and 103 between 1 and 100 femtoseconds (e.g., between 1 and 50 femtoseconds or between 1 and 20 femtoseconds).

Circuitry 107 and 108 may include, for example, regulation and control circuitry for UV-C, or other, sources of light on circuit board 101 as well as sources of light and other circuitry on other boards or external devices. Persons skilled in the art will appreciate that UV-C LEDs on circuit board 101 may be, for example, individually regulated and controlled or controlled as a group or in subsets. For example, circuit board 101 may include over ten (10) or over one hundred (100) UV-C LEDs. UV-C LEDs may be regulated and controlled in groups of two or more (e.g., three or more). A portion of UV-C LEDs may be regulated and controlled independently while another portion of UV-C LEDs may be regulated as a group or in sub-groups.

UV-C LEDs on printed circuit board 101 may be, for example, UV-C LEDs having the same wavelength of may have different wavelengths and they may be independently controlled at different times using different control profiles that provide different turn ON and turn OFF pulses (e.g., the duration of an OFF state for one or more UV-C LEDs may be the same duration or a different duration such as a longer or shorter duration than the ON duration for the respective one or more UV-C LEDs). The UV-C LEDs may all be between approximately 200 and 280 nanometers (e.g., provided at or between 250 and 270 nanometers such as provided at or between 255 and 265 nanometers) Some UV-C LEDs may be provided, for example, at or between 250 and 260 nanometers while others are provided, for example, at or between 260 and 270 nanometers. One or more additional light sources may be provided on board 101 such as, for example, UV-B, UV-A, VUV, and visible spectrum light sources.

Visible spectrum light sources may be provided, for example, to provide a visual indicator when board 101 is ON or OFF as well as different operating modes. For example, a visible spectrum LED may be a single-color LED (e.g., white, green, blue, or red) or a multiple color LED and may provide indication of when a battery (e.g., a rechargeable battery) is low and/or critically low on power. Manual inputs may be included on circuit board 101 to receive, for example, manual input to turn circuit board 101 ON, Off, and/or change between different modes of operation (e.g., different intensities for UV-C LEDs 102 and 103).

Circuit board 101 may be a single layer or multiple layer circuit board. For example, circuit board 101 may have two, three, four, or more layers. Printed circuit board 101 may be flexible. Persons skilled in the art will appreciate that a flexible circuit board may be at least partially or fully wrapped around or contorted around one or more objects (e.g., one or more working spaces for sterilization by the UV-C LEDs of board 101). Persons skilled in the art will appreciate that flexible circuit board 101 may utilized for multiple sterilization devices as flexible circuit board 101 may be able to flex around one or more objects (e.g., one or more hollow cylinders in which working material may be sterilized by UV-C LEDs) or may not be flexed and may lie flat next to an object (e.g., a surface of an object desired to be sterilized). Flexible circuit board 101 may be actuated so it can be flexed around different objects or placed next to an object so one device may be used in different configurations to change the location of elements of circuit board 101 to sterilize different objects and/or surfaces.

Circuit board 101 may include multiple rows and columns of UV-C LEDs and each UV-C LED, row of UV-C LEDs, and/or column of UV-C LEDs may be, for example, independently controlled (e.g., by processor 106 via additional circuitry such as additional circuitry 107). Circuit board 101 may include, for example, rows of three (or more) UV-C LEDs and columns of five (or more) UV-C LEDs). Persons skilled in the art will appreciate that rows may include the same number of UV-C LEDs or a different number of UV-C LEDs than other rows. Persons skilled in the art will appreciate that columns of UV-C LEDs may include the same or different number of UV-C LEDs than other columns. A row of UV-C LEDs may have, for example, six UV-C LEDs so that if circuit board 101 is rolled around a tube in a particular manner that the UV-C LED row provides a hexagonal disc around that tube. Each column may then, for example, provide another hexagonal disc of UV-C LEDs.

Persons skilled in the art will appreciate that circuit board 101 may be folded to provided UV-C LEDs facing in two (or more directions), left unfolded so the UV-C LEDs face in a single direction, wrapped around an object so the UV-C LEDs face into the object, folded inside of an object (e.g., a tube) so the UV-C LEDs face outside of the object, wrapped around an object (e.g., a bronchoscopy or probe) with the UV-C LEDs facing away from that object, or in any form to provide UV-C LED light to any object or objects. Persons skilled in the art will appreciate that circuit board 101 may have UV-C LEDs on a single side of board 101 or multiple sides of board 101.

Cross section 110 shows a cross-section of flexible circuit board 113 including UV-C LEDs 114 and 115 inside of a tube having an interior surface 112 and an exterior surface 111. Such a tube may be cylindrical in shape or may have a non-cylindrical shape. Any UV-C material utilized with a sterilization device may be UV-C transparent and may have UV-C transparency greater than fifty percent (50%), greater than seventy percent (70%), greater than eighty percent (80%), or greater than ninety percent (90%). Such a UV-C transparent material may be, for example, quartz. Cross section 110 may, for example, include a cross section that includes two or more UV-C LEDs such as three or more UV-C LEDS or six or more UV-C LEDs. Persons skilled in the art will appreciate that cross-section 110 may be provided such that a flexible circuit board having UV-C LEDs is inserted into a rigid or flexible tube that is UV-C transparent to be placed in a cavity of a living organism (e.g., a nasal, throat, or lung cavity) or wrapped around or a part of a structure (e.g., a bronchoscope, nasopharyngoscope, or another type of scope) in order to sterilize material placed about the tube having outer surface 111 and inner surface 112 from contaminants (e.g., viruses). Persons skilled in the art will appreciate that a thinner thickness between inner surface 111 and 112 of any tube used in connection with a sterilization device may provide more UV-C light to penetrate through inner wall 11 and 112 to interact with a working material. Accordingly, the thickness between inner surface 111 and 112 may be, for example, at or between half a millimeter and four millimeters (e.g., at or between half a millimeter and two and a half millimeters such as at or between a millimeter and two millimeters). For example, the thickness of a UV-C transparent material may be approximately two millimeters in thickness.

Side view 140 shows a side view of a cylinder with a flexible circuit board having UV-C LEDs wrapped around the cylinder. More particularly, side view 140 includes flexible circuit board 141 wrapped around a cylinder that has multiple UV-C LEDs such as UV-C LEDS 142, 143, 144, and 145. UV-C LEDs and 143 may be part of a UV-C disc that includes three or more UV-C LEDs. For example, the far side (not shown) of side view 140 may include a single UV-C LED aligned with UV-C LED 142 and 143 to provide a three UV-C LED disc around a hallow cylinder when placed around a hollow cylinder. UV-C LEDs may be facing into the hollow cylinder to provide UV-C light into a working area inside of the hollow cylinder in order to interact (e.g., sterilize) material (e.g., virus) in and/or moving through that working area. UV-C LED 142 may be aligned with UV-C LED 144 and UV-C LED 143 (and other UV-C LEDs) may be aligned with 145 (and other UV-C LEDs), respectively, so that the UV-C LEDs of multiple discs and/or rows are aligned with each other when wrapped around an object.

Cross-sectional view 120 shows circuit board 123 that may include one more UV-C LEDs (e.g., UV-C LED 124) located around a UV-C transparent hollow cylinder provided by interior wall 121 and exterior wall 122.

Cross-sectional view 130 shows circuit board 131 located around a hollow cylinder that included an interior wall 132 and an exterior wall 133. Circuit board 131 may have one or more UV-C LEDs (e.g., UV-C LEDs 134 and 135).

Side view 150 shows flexible circuit board 152 wrapped around a hollow cylinder such that LED discs are formed that are staggered from one another. For example, UV-C LED 153 may be associated with two or more UV-C LEDs located on the far side of the cylinder while UV-C LEDs 152 and 154 may be associated with one or more UV-C LEDs located on the far side of the cylinder. Each UV-C LED disc may have the same (or different) number of UV-C LEDs but, for example, these UV-C LED discs may be staggered such that material flowing through the cylinder at different locations may have staggered UV-C LEDs that may be closer to the material than if the UV-C LEDs were not staggered with respect to one another. Persons skilled in the art will appreciate that multiple UV-C discus, rows, or columns may be staggered in two or more configurations (e.g., three or more configurations) and multiple groups of UV-C LEDs may be staggered differently than different groups of UV-C LEDS.

Device 160 shows a stepped hollow cylinder 162 that has three circuit boards, each having multiple UV-C LEDs wrapped around different portions of the stepped hollow cylinder. For example, circuit boards (e.g., circuit board 101 of FIG. 1) may be placed (e.g., wrapped around) portions 162, 163, and 164. Persons skilled in the art will appreciate that multiple circuit boards (e.g., circuit board 101 of FIG. 1) may be independently controlled via the same of different firmware on each board. Multiple circuit boards may be coupled to a processor and/or circuit board located outside of the boards with UV-C LEDs. A circuit board with UV-C LEDs may act as a master control circuit board to another circuit board with UV-C LEDs that acts as a slave circuit board such that the master control circuit board controls the slave circuit board.

Cross-sectional view 170 includes circuit board 173 around a hollow cylinder including interior wall 171 and exterior wall 172. The cylinder, as in any structure that is provided to include a working space in that structure, may be UV-C transparent. Circuit board 173 may include one or more UV-C LEDs (e.g., UV-C LED 176) that faces into the walls 171 and 172 such that UV-C light from UV-C LED 176 passes through walls 172 and 172 to impact the working space provided by wall 171. A material, e.g., air, may be flowed through the working space provided by wall 171 so that UV-C LEDs may impact (e.g., sterilize) that material from contaminants (e.g., virus and/or bacteria). Persons skilled in the art will appreciate that a flexible circuit board having UV-C LEDs may be laminated into the hollow cylinder itself (e.g., between walls 171 and 172. Such a configuration may, for example, provide UV-C LEDs closer to the working space. A fan, or other material movement system, may be provided to impact the speed that material is moving through the working space.

Post 175 may be UV-C transparent and may include UV-C LED 174. Configuration 181 may be provided in place of UV-C 174 and may include multiple UV-C LEDs. Any UV-C LED may be tilted at an angle on any axis in order to provide UV-C LED light in any direction. UV-C LEDs 182, 183, 184 may be provided on structure 185 and may be tilted differently on one or more axis from each other).

UV-C LEDs 174 or any UV-C LED located outside of a circuit board (e.g. circuit board 173) may be communicatively coupled (e.g., coupled by a physical conductor) to circuit board 173 so that circuit board 173 may control one or more UV-C LEDs located outside of circuit board 173.

A working space may be any working space in any device such as a ventilator device. In providing UV-C sterilization in a ventilator device any air flowing through that ventilator device (e.g., air entering, flowing through, or exiting) the device may be sterilized.

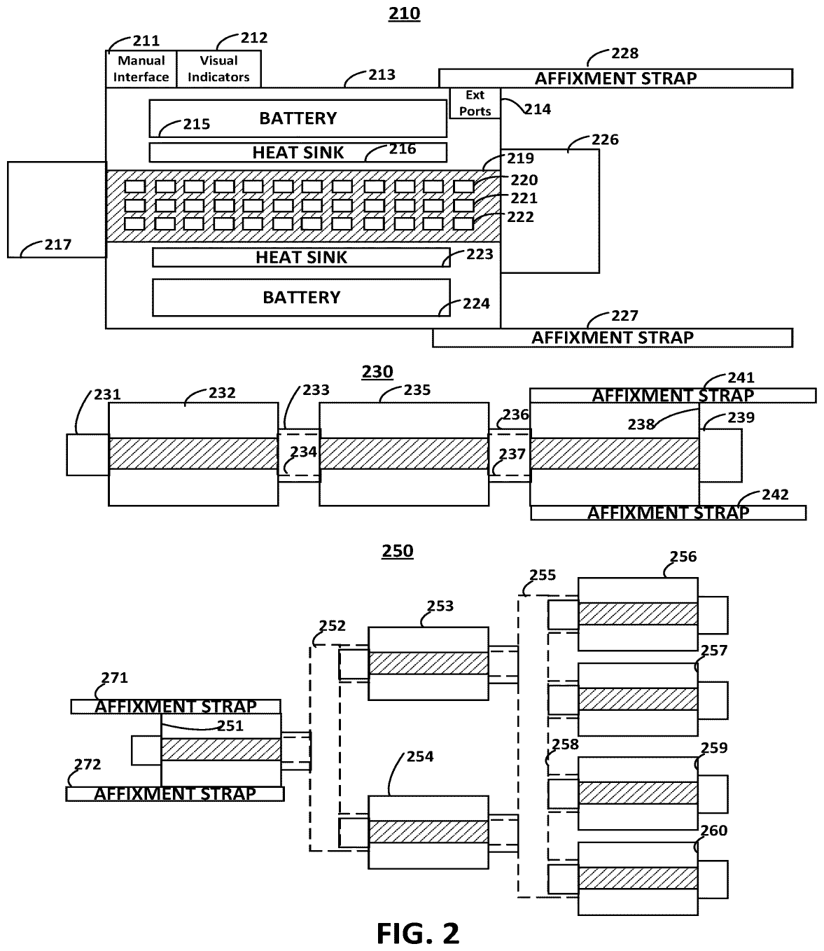

FIG. 2 shows device 200 that may include housing 213 that may include affixment straps 228 and 227 that may, for example, affix device 200 to a user (e.g., a user's head so that a user may breath through device 200). A hollow cylinder may be fluidically coupled to mateable portion 217 and mateable portion 218 so that a working substance (e.g., air in a ventilator) may pass through mateable portion 217, through the cylinder, and through mateable portion 218. Mateable portion 217 may be a male mateable part that fits into female mateable part (e.g., mateable part 218 may be a female mateable part). In doing so, tubing used in, for example, medical devices such as ventilators may be coupled to mateable portion 217 and 218 such that a working substance flowing through the ventilator is temporarily redirected through device 210. Circuit board 219 may include UV-C LEDs (e.g. UV-C LEDs 220, 221, and 222) around a cylinder that circuit board 2019 is wrapped around). One or more heat sinks (e.g., heat sinks 216 and 223) may be wrapped around a portion or all of circuit board 219 to draw heat generated from circuitry and UV-C LEDs away from the working space (e.g., the space inside of the cylinder) The cylinder may be a UV-C transparent material (e.g., quartz) and may include a thickness between an inner wall and an outer wall between approximately 1.5 millimeters and 2.5 millimeters (e.g., approximately 2 millimeters). Persons skilled in the art will appreciate that heat sink 210 and 223 may be a single heat sink wrapped around circuit board 219 wrapped around a hollow cylinder (or other structure providing a working space). Persons skilled in the art will appreciate that a cylinder or other structure may not be provided and circuit board 219 may define the working space itself. For example, circuit board 2019 may be wrapped into a cylinder and a working material may be followed through that cylinder. A protective layer may be placed (e.g., sprayed or placed) on one or more portions of one or more surfaces of the circuit board to provide protection for the circuit board from any working material.

Device 210 may include one or more batteries 215 and 224. Persons skilled in the art will appreciate that batteries 215 and 224 may be separate batteries or a single battery wrapped around housing 213. Batteries may be rechargeable or permanent and removable and replaceable. Charging circuitry may be provided. External power may recharge the power or, for example, may power circuitry of device 210 directly. Switching and regulation circuitry may control, for example, when external power (e.g., wall power) is utilized to charge a rechargeable battery and/or power circuitry of device 210 directly. Manual interfaces 211 may be included such as, for example, to turn device 210 ON/OFF and or change modes or enter other input data into device 210 (e.g., configure device settings and or device modes). Visual indicators 212 may be a bi-stable or non bi-stable display and/or single-color light source (s) and/or multiple color light source(s). A visual indicator may be a two-color display (e.g., black and white or two tone display) or a several color display (e.g., a color display) and may include an interface for the consumer. Visual indicators 212 may include the status of device 210. Status may include, for example, status information such as, for example, whether device 210 is operating properly or incorrectly as well as data associated with the device. For example, device 210 may provide a visual indication of a low battery, broken part (e.g., broken UV-C LED). Audio indicators may also be provided such as speakers. Audio and/or visual information may be provided such as, for example, when a battery is less than a particular amount of charge (e.g., less than twenty percent or less than ten percent of charge) or when a software update is available. External ports 214 may be provided anywhere on housing 213 such as on mateable port 217 and 218 such that external power and/or control and/or data input/output may be provided. By including external ports 214 on mateable portions multiple devices can be physically coupled together and the coupled devices may communicate to each other (e.g., control and power each other). Any number of devices 210 may be coupled to one another to, for example, provide a multiple or several device array or, for example, to increase the sterilization impact on a working substance. Two or more devices 210 may be coupled to a ventilator. Two or more devices 210 may be coupled to different parts of a ventilator or may be coupled adjacently to a single part of a ventilator.

Devices 230 that may include affixment straps 241 and 242 that may, for example, affix device 200 to a user (e.g., a user's head so that a user may breath through device 200). Devices 230 are provided that include device 232 having mateable portions 231 and 233, device 235 having mateable portions 234 and 236 and device 328 having mateable portions 237 and 239. A working substance can be flowed (e.g., pushed and/or pulled) through an opening in mateable portion 231 and through devices 232, 235, and 238 to be expelled through an opening in mateable portion 239.

Devices 250 may include affixment straps 271 and 272 that may, for example, affix device 200 to a user (e.g., a user's head so that a user may breath through device 200). Devices 250 may be provided and may include devices 251, 253, 254, 256, 257, 259, and 260. Adaptors 252 and 255 may be included to create a joined working space between any number of devices. Adaptor 252 may, for example, fluidically couple device 251 to device 253 and 254. Adaptor 255 may, for example, fluidically coupled devices 253 and 254 to devices 256, 257, 259, and 260.

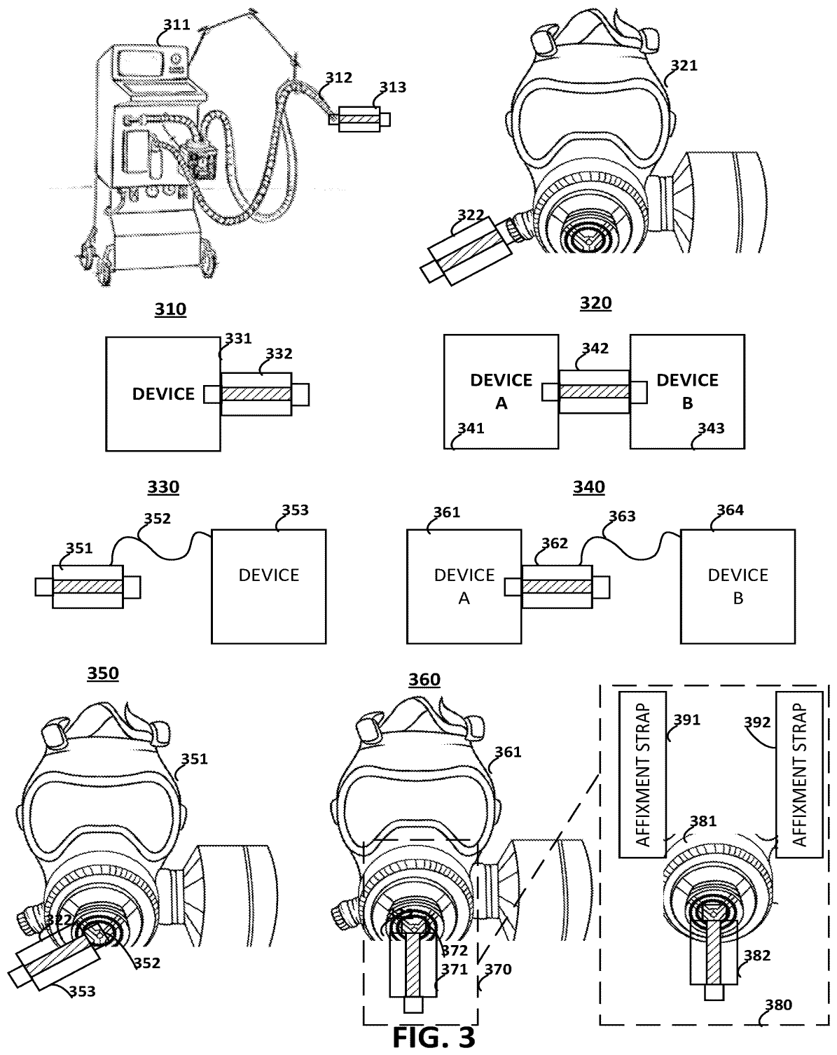

FIG. 3 shows ventilator 310 that may include housing 311, tubing 312 and device 313 that may include device 313 for providing UV-C light to the working substance provided by tubing 312. Deice 313 may be, for example, any UV-C generating device included herein such as, for example, device 100 of FIG. 1.

Persons skilled in the art will appreciate that a UV-C generating device may have liquid and/or gas flowed through it from any structure. Accordingly, for example, a UV-C sterilization device may be placed about an input and/or output and/or filter port to any device such as a facemask. Accordingly, for example, a facemask wearer (e.g., a military, police, firefighter, caregiver) may enjoy improved protection against contaminants (e.g., bacteria and/or virus). Configuration 320 may be provided that may include UV-C sterilization device 322 fluidically coupled to an air channel of mask 321. Persons skilled in the art will appreciate that multiple UV-C sterilization devices may be coupled to one or more air channels of mask 321.

Configuration 330 of FIG. 3 shows device 331 coupled to UV-C generating device 332. Device 331 may be, for example, a substance cooler, substance heater, substance fan, and may be fluidically coupled to provide the substance worked on, expelled, or input into device 331 through device 332 to provide, for example, sterilization capability.

Configuration 340 may be provided any may include device 341 fluidically coupled to device 343 through UV-C generation device 342 such that a substance moved between device 341 and 343 may be sterilized by, for example, device 342.

Configuration 350 may include device 353 communicatively coupled to UV-C generating device 351 via physical or wireless communications 353 such that information and controls may be provided between device 353 and device 351 through filter port 352. Persons skilled in the art will appreciate that a UV-C inactivation device may be attachable and removable from a mask (e.g., a satellite mask) so that the inactivation device may be replaced without having to remove a mask or one or more inactivation devices may be integral to a mask (e.g., a satellite mask) and not removable from the mask while the mask, for example, is being worn by a user.

Configuration 360 may be included that includes device 361 that may be fluidically coupled to device 371 through structure 372 and communicatively coupled to device 371. Device 361 may be a full-facemask and device 381 of mateable mask 380 may be mateable to device 361 such that a full facemask may be provided when mated and a light-weight device may be provided by device 380 when unmated. Affixation straps 391 and 392 may be utilized, for example, to attach device 381 to a user's head. Inactivation device 382 may be a portion of device 380 such as, for example, an integral portion when device 380 is head worn or a removable portion when device 380 is head-worn.

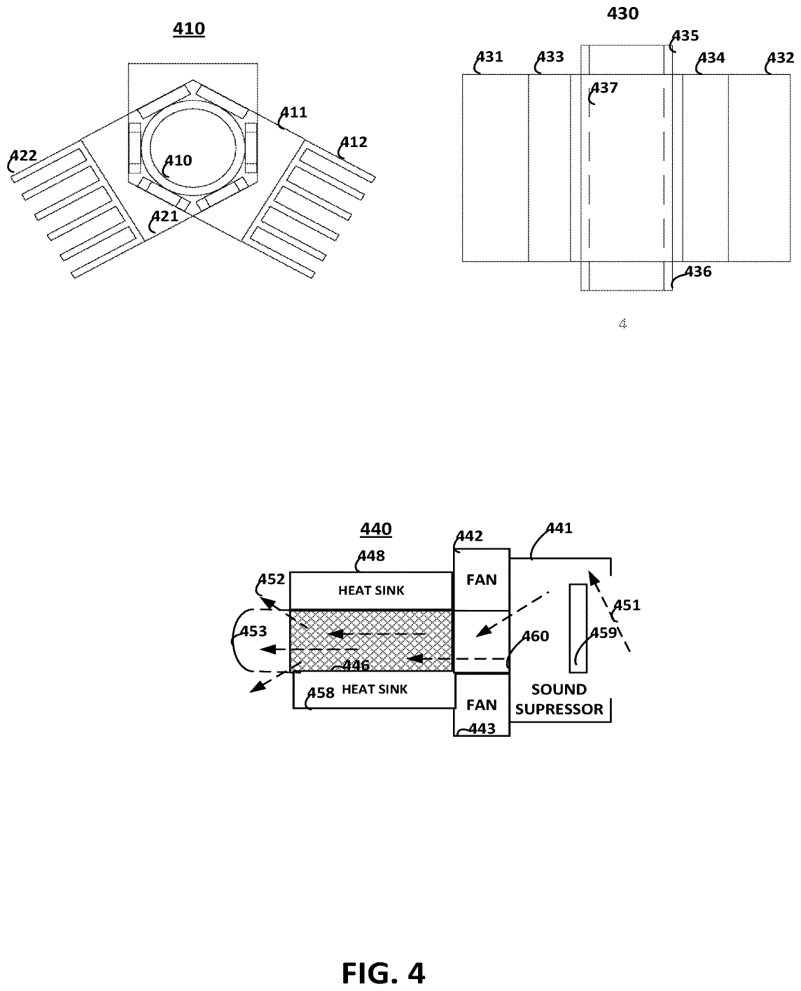

FIG. 4 shows device 410 that may include inactivation working area 410 that may draw heat through heat sinks 411 and 421 and the heat may be carried away via air using fans providing air across heat fins 422 and 412 or not using fans providing air across heat fins 422 and 412. Person skilled in the art will appreciate that UV-C light sources may be placed outside of working area 410 and may provide energy through UV-C portions of area 410 (e.g., apertures cut into a UV-C reflective cylinder and sealed with UV-C transparent material such as quarts).

Device 430 may be, for example, a perspective view of device 410 and may include heat sink fins 431 and 432, heat sinks 433 and 434, inactivation area 437, and mateable air channels 435 and 436 that may, for example, mate with different structures (e.g., masks, mateable ports, filters, etc.).

Persons skilled in the art will appreciate that a UV-C working area may be provided by a cylinder or other hollow structure such as a spherical cylinder, elliptical cylinder, rectangular cylinder/prism, square cylinder/prism, triangular cylinder/prism, or any other shape channel including channels that may change shape as the channels progress in a direction. UV-C LEDs may be provided on a flexible printed circuit board that is flexed around a cylinder (e.g., a quartz cylinder) and mounted to the cylinder and/hour housing (e.g., through screw apertures located on the printed circuit board). Any number of rows and columns of UV-C LEDs may be provided and these rows and/or columns may be aligned and/or staggered for entire columns and/or rows or portions of columns and/or rows.

One or more heat sinks may be provided, for example, on the back of a flexible circuit board so that heat from a UV-C LED may travel from the UV-C LED through the circuit board to one or more heat sinks. A heat sink may be for example, aluminum and/or copper (e.g., copper inside of the aluminum to improve flow of heat through the aluminum). Thermal paste or another thermal substance may be utilized to improve thermal coupling of a portion of a device (e.g., back of circuit board under a UV-C LED) with a heat sink. One, two, or several Heat dissipation fins, such as fins 402 and 419, may be provided and may be provided as part of or coupled to one or more heat sinks. Persons skilled in the art will appreciate that batteries may be provided in air sanitization houses.

An air sanitization device may be provided in which an object may be passed through one or more UV-C working area(s). Different types of UV light sources (e.g., tube lamps) and different types of UV light (e.g., UV-A and/or UV-B devices) may be provided to provide various types of UV light into a UV working area.

Persons skilled in the art will appreciate that a UV-C generation device may have any number of UV LEDs of any number of types and wavelengths and be provided in any configuration and density. Multiple devices may be fluidically coupled together o so that the sterilization capability may be increased by creating additional UV-C working areas that are fluidically coupled together (e.g., the output of an air sanitization device is coupled to the input of an air sanitization device.

A UV-C working area defining structure (e.g., tube) may be provided at a slant with respect to a base. In providing a slant, UV light (e.g., UV-C light) may be directed away from an opening so that UV-C light does not pass through the opening (e.g., the entrance). Different mating structures may be provided about input and/or output outlets of an air sanitization device so that the air sanitization device may be, for example, coupled to an external device such as a ventilator for air sterilization.

A conveyer or moveable tray or pushing object may be utilized to move an object through a working channel. Persons skilled in the art will appreciate that structures may be provided in a UV working area to slow down an object and or direct an object in a certain direction in order to, for example, increase the time of an object in a working channel. For example, a working channel may include multiple turns in order to, for example, potentially decrease the speed of objects flowing through a working channel.

Persons skilled in the art will appreciate that the entrance and/or of a UV working area may take any dimension and shape, may take the same dimension and/or shapes, and/or may take different dimensions and/or shapes. Furthermore, persons skilled in the art will appreciate that a UV working area may have multiple entrances and multiple exits (and may be bi-directional do objects can enter from any exit and enter through any exit). The working area channel may have the same dimensions or different dimensions as an opening. Multiple or several connected and/or independent UV working areas may be provided in a device.

An opening to a UV-C working area may, for example, have any length and/or width. For example, the width of an opening may be less than, greater to, or equal to 0.5 inches, 1.0 inches, 1.5 inches, 2.0 inches, 2.5 inches, 3.5 inches, 6 inches, 12 inches, 18 inches, 24 inches, etc. For example, the length of an opening may be less than, greater to, or equal to 0.5 inches, 1.0 inches, 1.5 inches, 2.0 inches, 2.5 inches, 3.5 inches, 6 inches, 12 inches, 18 inches, 24 inches, etc. For example, the width of an opening may be less than 6 inches and the length of an opening may be less than 24 inches.

Device 440 may include sound suppressor 441 in which air is brought into a sound suppression chamber to block sound coming from either side of sound suppression structure 441. Sound suppressor may include one or more inlet sound barriers 459 that may be fabricated from a plastic (e.g., a 3D printed plastic and/or a molded plastic). A soundproofing material (e.g., a soundproofing foam) may be provided in the device between fan 442 and structure 459.

Fans 442 and 443 may be provided in any number of structures. For example, Fans 442 and 443 may be provided. Any number of fans may be provided in fans 442 and 443 (e.g., a pair of fans). Each pair of fans may be, for example, count-rotating compression fans or may be non-compression fans. Each pair of counter-rotating fans may be, for example, synched together or may be offset from one another. Fans may be utilized to push air through a device (e.g., a heat sink and/or heat sink fins) and/or pull air through a device (e.g., during different operating modes).

A UV-C working area may be provided as area 446 which may be defined, for example, by a UV-C reflective structure such as a UV-C reflective tube. Apertures may be provided in the tube for UV-C light sources (e.g., UV-C LEDs) to be included through one or more circuit boards (e.g., flexible circuit boards) or other structures. Transparent windows may be placed between the UV-C LEDs and the working area so that working area substances (e.g., air) does not touch the UV-C LEDs. Such windows may be UV-C transparent (e.g., a quartz or other UV-C transparent material). Heath sinks (e.g., copper heat sinks) and heat sink fins (e.g., aluminum heat sink fins or copper heat sink fins) may be utilized to remove heat from UV-C LEDs. UV-C inactivated air from working area 446 may be routed out of the UV-C inactivation chamber and returned over the exterior of a structure providing working area 446 (e.g., through heat sinks and heat sink fins removing heat from UV-C light sources providing UV-C light in working area 446). In doing so, for example, inactivated air may be utilized to, for example, also cool the device by removing heat from the heat sinks. In doing so, for example, only inactivated air may be moved out of the device. In doing so non-inactivated air is not moved out of the device so virus in non-inactivated air may not be spread by the device. Furthermore, inactivated air that is removing heat may be routed out of device 440 (e.g., routing 452). Furthermore, curved surface 453 (e.g., which may be a convex surface) may diminish UV-C slight from inactivation chamber 446 from leaving the device and may reduce air resistance out of the device to increase airflow through a device. Person skilled in the art will appreciate that the air resistance of the device may be, for example, the air resistance of working area 446 and structure 453 may reduce air resistance further by providing a less resistance path to flow over as it exists device 440. Housing of device 440 may be a metal, which may increase sound absorption, and then have a plastic casing outside of that metal, which may reduce the amount of heat a user feels if the person touches the outside of housing of device 440. Housing may be a metal (e.g., an aluminum). Housing may be, for example, a plastic (e.g., a 3D printed or molded plastic).

Fan 442 may provide air past heat sink 448 and fan 443 may provide air past heat sink 458. Fans 442 and 443 may, for example, be utilized to also push air through a working area through, for example, valves that may be controlled to move air through a working area such that resistance of additional filters added to a working channel may be reduced by supplemental air. Persons skilled in the art will appreciate that fans 442 and 443 may pull air through working area 446 such that, for example, inactivated air is brought to a user and pushed through area 446 such that exhaled air is moved through area 446.

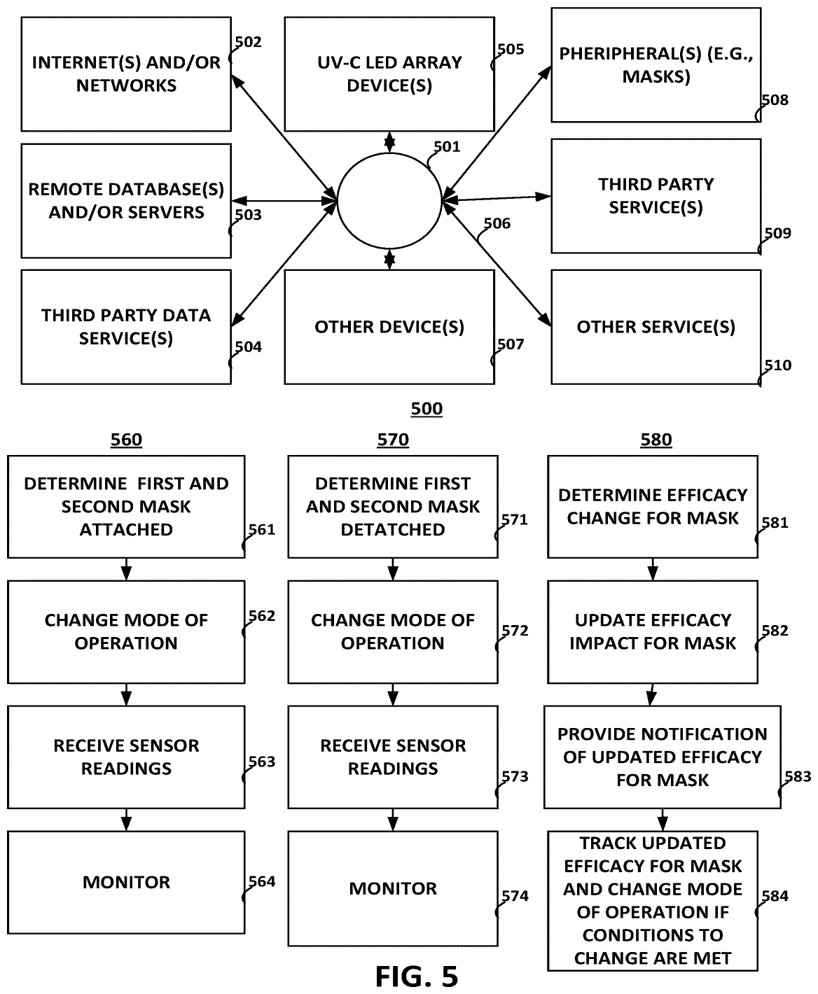

FIG. 5 shows topology 500 that may include UV-C generating devices 205 that may include one or more UV-C arrays of LEDs coupled through communications 501 to one or more internets and/or networks 502, one or more remote databases and/or servers 503, one or more third party data services 504 (e.g., medical data services for a patient utilizing a UV-C generating device), one or more other devices 507 (e.g., one or more other medical devices for a patient using a UV-C generating device), one or more other services 510 (e.g., a service that provides data regarding other UV-C generating devices), one or more third party services 509 (e.g., timing/clock services for the timing/clock of a UV-C generating devices), and/or one or more peripherals 508 (e.g., masks, external displays, external batteries).

Persons skilled in the art will appreciate that UV-C generation devices may be utilized for surface sanitization such as sanitization of organic or inorganic material.

Process 560 shows step 561 which may include a determination if a mask is attached. New air speed may be determined in step 562. Such a new airspeed may be achieved by, for example, detecting an object and retrieving the airflow speed for that object when connected. Alternatively, for example, airflow speed may be determined by detecting airflow speed. The new airflow speed determined in step 562 and implemented in step 563. A device may monitor for the attachment or detachment of a structure and/or a change in airspeed in step 564.

Process 570 may be included and a device may determine a change in environment. A change in environment may be detected, for example, in a change in airspeed, fan resistance, humidity, temperature, pressure, elevation, or any other metric. Device, such as a mask, may determine to change operation in step 4572 and implement the change in operation in step 573. The environment may continue to be monitored in step 574.

Process 580 may be included and may include step 581 in which an efficacy change is determined for a device such as, for example, one or more masks. An efficacy may be, for example, associated with an inactivation rate at a particular airspeed and/or in a particular environment (e.g., particular humidity and/or elevation) for a particular virus. An efficacy change may be determined, for example, if an airspeed changes and efficacy may be updated in step 582 and a notification of an updated efficacy may be provided in step 583 and updated efficacy may be tracked in step 584. An efficacy notification may be, for example, a visual notification representative of an efficacy (e.g., a range of inactivation such as an inactivation of a particular virus between two inactivation rates or at a particular inactivation rate).

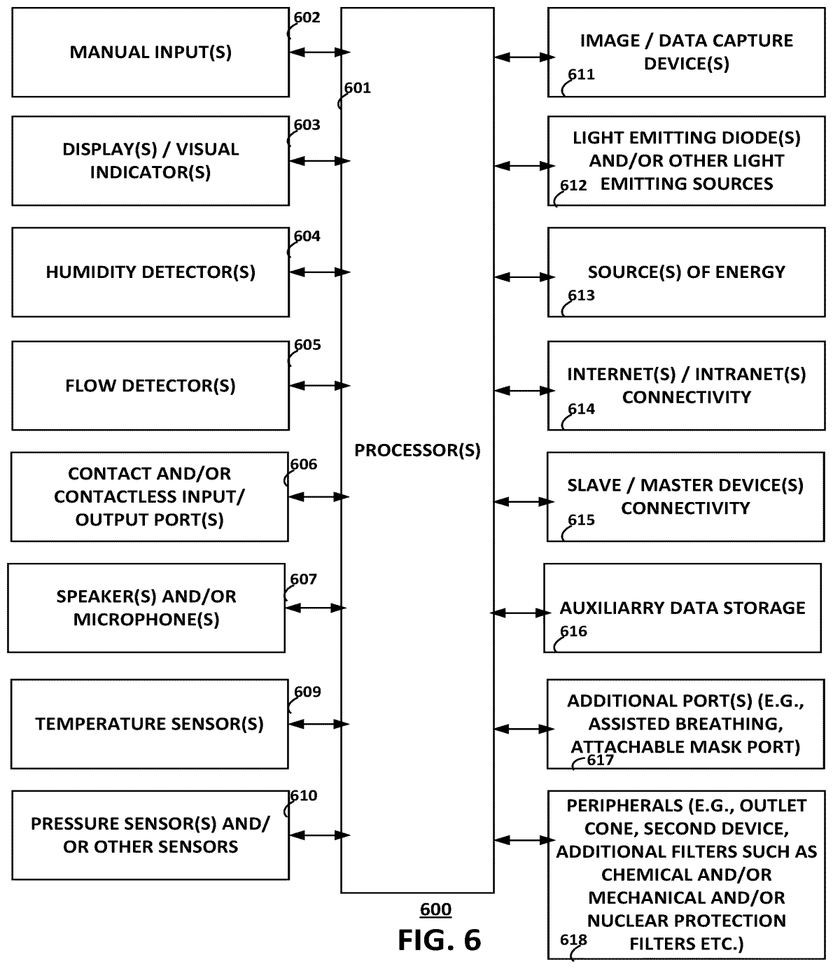

FIG. 6 includes device 600 that may include one or more processors 601, one or more manual inputs 602, one or more displays and/or visual indicators 603, one or more humidity detectors 605, one or more flow detectors 605, one or more contact and/or contactless input and/or output ports 606, one or more speakers and/or microphones, one or more temperature sensors 6oi (e.g., to sense temperature in a working space), one or more pressure sensors 610 (e.g., pressure sensing for sensing pressure in a working space) and/or other sensors (e.g., metal sensors UV-C transparency sensors), one or more image and/or data capture devices 610 (e.g., a visible and/or infrared or other spectrum camera or data capture device), one or more light-emitting diodes and or other light emitting sources 612 (e.g. UV-C LEDs and/or UV-C light emitting sources), one or more sources of energy 613 (e.g., rechargeable and/or removable batteries), one or more internet or intranet connectivity devices 614, one or more slave and/or master devices 615, one or more auxiliary data storage devices 616 (e.g., a remote server), and one or more peripherals 618 (e.g., an outlet cone such as an annulus cone or a second inactivation device or an air funnel, a second device such as a second mask, one or more filters such as multiple filters in series such as one or more chemical, mechanical, and/or nuclear protection filters). Persons skilled in the art will appreciate that different structures (e.g., air cones and air funnels) may be detected by detecting a change in airflow pre-determined for that particular structure and/or a mechanical, electrical, and/or other detection of the attachment of an object. For example, an object may have a particular mechanical structure that may enable a particular switch in an inactivation device to activate when the object is mated properly with the inactivation device.

Peripheral 618 may include, for example, any number of additional inactivation devices, or other sanitization devices, such as an air inactivation device on an oscillator, such as a two axis, three axis, or more than three axis oscillator for moving a UV-C generating device around the controllable various axis. One or more sound suppression structures, such as sound suppression cambers that air flows through, may be permanently fixed to a device and/or removably attached to a device. In doing so, for example, a user may purchase and add sound suppression structures to increase the sound suppression of the device. Multiple sound suppression structures may be mated together through mateable structures to provide additional sound suppression. Additional ports 617 may be provided and may include ports such as removable from and attachable to ports for assisted breathing (e.g., fans for assisted breaching), attachable masks, attachable covers, attachable face seals (e.g., foam, air pillow, gel, rubber, polymer, etc.).

Persons skilled in the art will appreciate that any type of UV light may be utilized, for example, to create a UV inactivated vaccination and that particular strains may be inactivated with one wavelength of UV and another strain may be inactivated with a different wavelength. Such inactivated virus vaccinations may be aerosolized and may be inactivated in aerosolized forms. An aerosolized vaccination may then be, for example, changed to be in a different form (e.g., a liquid vaccination). Persons skilled in the art will appreciate that a UV-C inactivated strain vaccine may include portions of light outside of UV-C and a majority of the light (e.g., 50 percent or more, 75 percent or more, 85 percent or more, 90 percent or more, 95 percent or more, 98 percent or more, 99 percent or more, or 100 percent) may be UV-C.

Persons skilled in the art will appreciate that a UV-C inactivation device may include eighteen UV-C LEDs and may inactivate bacteriophage, a DNA virus, at 30 liters per minute at an inactivation rate greater than 99.999% at a humidity greater than 50%. Persons skilled in the art will appreciate that a UV-C inactivation device may include eighteen UV-C LEDs and may inactivate, SARS-CoV-2, a RNA virus, at 400 liters per minute at an inactivation rate greater than 99% at a humidity greater than 50%.

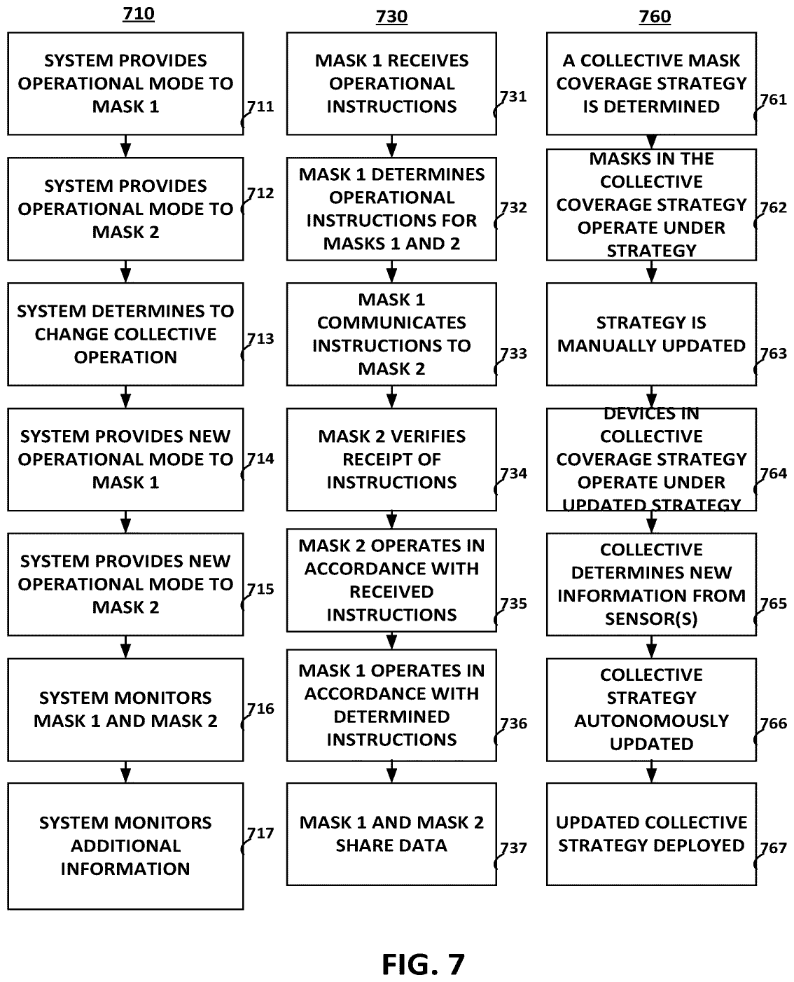

FIG. 7 includes process 710 that may include step 711, in which a safety system may provide an operational mode to a sterilization device such as, for example, an air inactivation device such as a mask (e.g., a multiple piece mateable mask). Step 712 may be included in which the safety system provides an operational mode to a different sterilization device such as, for example, another air inactivation device. Person skilled in the art will appreciate that any number of sterilization, inactivation, and/or sanitization devices may be controlled by a system and/or may collectively work together to execute a strategy in order to clean an environment (e.g., a room, building, or other area) to make the area cleaner (e.g., reduce the viral load in the environment). The system may receive information from different devices (e.g., the device in step 711 and/or the device in step 712) as well as external sources and/or manual input to determine a change in collective operation in step 713 of one or more devices. The system may provide a new operational mode to the first device (or devices) in step 714 and a new operational mode to a second device (or devices) in step 715. The new operational modes may be the same and/or different for each device or a particular set of devices.

Process 730 may include step 731 in which a device (e.g., a face mask) receives and/or determines operational instructions and step 732 where the device determines the operational instructions for itself and at least one other device that it is in communication with. The device communicates instructions to the other device or devices in step 733 and the other device or devices verify receipt of instructions in step 734. The other device or devices operate in accordance with the received instructions in step 735 and the master device operates in accordance with its determined instructions in step 736 and the master and slave devices share data in step 737. Persons skilled in the art will appreciate that a device that is a master device may switch master status with another device. For example, a device may be determined to be a master device if, for example, it is a device in a certain area or that has certain access to information or any number of attributes (e.g., it is the device that is operable to communicate with the highest integrity with a particular system or device or number of devices such as number of inactivation devices).

Process 760 may include a collective coverage strategy for area cleaning devices (e.g., multiple mateable masks). A collective strategy may be determined in step 761, devices in the collective coverage strategy may operate under the strategy in step 762, the collective strategy may be manually updated in step 763, the devices may operate under the updated strategy in step 764, the strategy may be autonomously updated in step 766 (e.g., based on pre-determined strategy decision rules based on information from one or more devices in the collective of devices, and the updated collective strategy may be deployed in step 767. Persons skilled in the art will appreciate that devices may be provided with a priority such as to protect individuals or protect areas. In an individual protection strategy, for example, individuals may be tracked and air and surface inactivation devices may be positioned to spend more time cleaning surfaces around (e.g., in the area an individual is more likely to interact with) and air around the individuals). Accordingly, for example, tracking may determine how one or more individuals are moving in an area and air inactivation devices may provide inactivated (e.g., cleaned) air where the individuals are located and/or where the individuals are expected to move toward.

An inactivation device (e.g., a SARS-CoV-2 inactivation device) may include a fan portion and working area portion. A working area portion may include, for example, a structure that provides a working area for air, or another substance, to flow through, one or more circuit boards provided about the structure that includes one or more UV-C light sources (e.g., LEDs) as well as additional electronics (e.g., microprocessors, input/output ports, additional circuitry), heat sinks and heat sink attachment structure(s) (e.g., thermal paste), heat sink fins and heat sink fins attachment structures (e.g., if the heat sink is separate from the heat sink fins such as a copper heat sink and aluminum heat sink fins), and/or a primary housing that provides a mechanical structure as a foundation for the placement of structures in a working area portion.

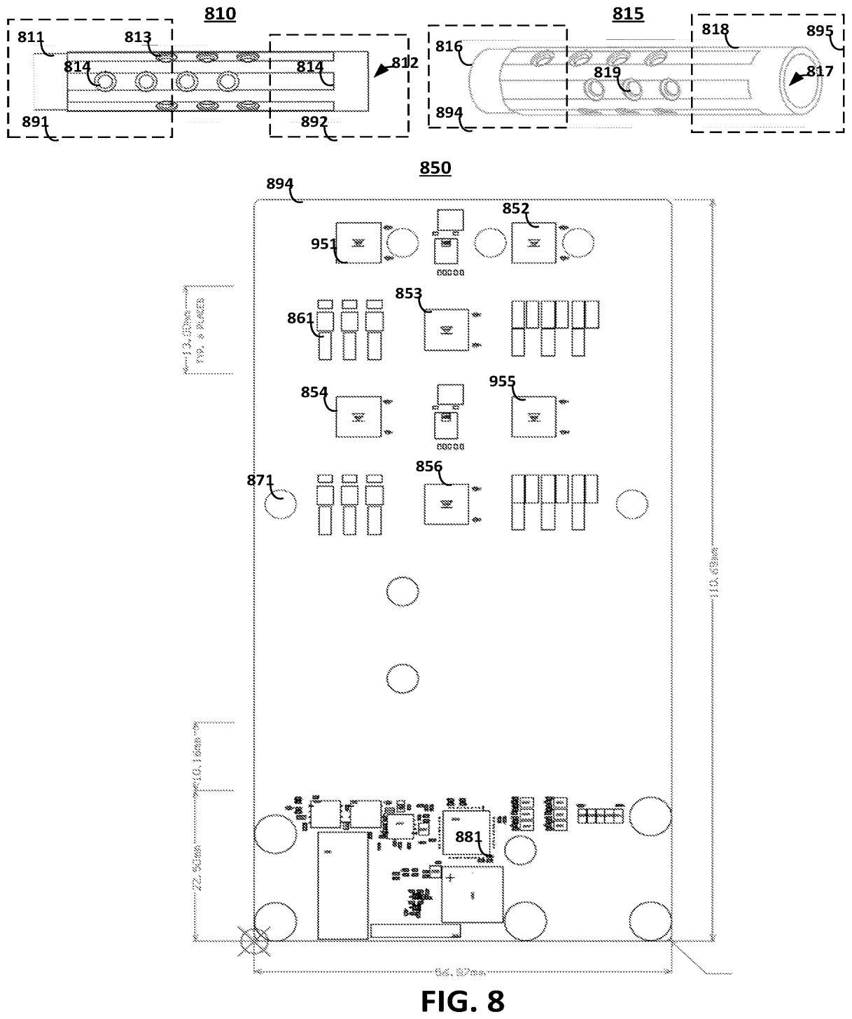

FIG. 8 shows tube 810 that may provide a working area for UV-C inactivation. Tube 810 may include mateable portion 811 and air opening 812. Apertures (e.g., stepped apertures to receive UV-C transparent windows, such as quartz, in the step of an aperture and fixed using an adhesive) may be provided such as aperture 813. A UV-C LED may be provided to provide UV-C light into one or more UV-C apertures on tube 810. Multiple (e.g., eighteen or more) UV-C LEDs may be provided on a flexible circuit board and wrapped and attached (e.g., via a mechanical fixation device(s) such as screw(s) or adhesive(s)) to the tube. Trough 814 may be provided to permit a circuit board to fall into trough so that UV-C LEDs may get closer to a working surface (e.g., to increase intensity and inactivation). Additional apertures (e.g., aperture 814 may be provided for sensors such as, for example, light sensors (e.g., to determine the intensity or other attributes of one or more UV-C LEDs).

Tube 815 may be, for example, a different perspective view of tube 810 of FIG. 8. Tube 815 may be fabricated, for example, completely, or partially, from a UV-C reflective material (e.g., a PTFE material) and may have UV-C reflectivity greater than 95% (e.g., greater than 95% for nanometer wavelength ranges centered at a point in the range of 250 nm and 280 nm such as centered at a point in the range of 255 nm to 270 nm such as centered at a point in the range of 255 nm to 265 nm). Tube 815 may include external surface 818, mateable portion 816, and air opening 817.

Tube 810 may be shorted by removing portions 891 and 892 so that, for example, a decreased sized inactivation device may be provided as part of a decreased size mask (e.g., a partial face mask). Tube 815 may be shorted by removing portions 894 and 895 so that, for example, a decreased sized inactivation device may be provided as part of a decreased size mask (e.g., a partial face mask).

Persons skilled in the art will appreciate that fans may be provided in a device and may provide airflow at rates above 400 liters per minute (e.g., above 500 liters per minute). Multiple circuit boards with UV-C arrays may be placed around multiple tubes. Tubes of any width and fans of any width may be provided. For example, four flexible circuit boards may be provided about one or more in-line tubes that may have inner diameters between twenty-five and fifty millimeters (e.g., between 30 and 33 millimeters0 and air may be driven through the working area to inactivate pathogens at speeds of at least 1,000 liters per minute, at least 1,500 liters per minute, or at least 2,000 liters per minute.

A substance inactivation device may include heat sink fins (e.g., aluminum fins) coupled to heat sinks (e.g., copper and/or aluminum heat sinks such as an aluminum heat sink with copper heat transportation structures such as rods within the aluminum). Heat sinks may be a heat sink structure that couples to, for example, a flexible circuit board coupled to tube 825. Tube 825 may have a different shape on its external surface (e.g., a six sided shape) than the shape on its internal surface (e.g., a spherical cylinder). A tube for defining a working area may be fabricated, for example from a UV-C reflective material (e.g., PTFE) and may have apertures for placing UV-C transparent materials (e.g., quartz) so UV-C light from UV-C LEDs on a flexible circuit board placed on the exterior of the tube may flow through the UV-C transparent materials and enter a working area provided by the tube. Person skilled in the art will appreciate that the number of sides on the external surface of a tube providing a working area) may match the number of UV-C LED locations that are provided about the perimeter of that tube. For example, if there are six possible UV-C LED locations about an external surface perimeter of a tube providing a working area then that tube may have six sides on the external surface. Persons skilled in the art will appreciate that the external surface of the tube may be any shape (e.g., spherical) and may match the shape of the internal surface of that tube. A tube may be fabricated from multiple materials such as, for example, a tube of UV-C transparent material (e.g., quartz) that is coated (e.g., either on its interior or external surface) with a UV-C reflective material (e.g., aluminum) with spaces in the UV-C reflective material aligning with UV-C locations. Structures may be provided and may be utilized to provide a mechanical support structure for attaching pieces. A structure may also be, for example, a heat sink. A portion that generates or conducts heat may be provided with or without heat sink fins. Additional heat sink or heat sinks may be provided and may attach to such a portion. A heat sink may thermally couple to one or more sides of a flexible circuit board, or other structure as a non-flexible circuit board, that provides UV light sources (e.g., UV-C LEDs). For example, a heat sink may be thermally coupled to UV sources located on two sides of the exterior of a tube providing a working area. Any number of screw and/or mounting holes and/or structures may be provided on any structure of a substance sanitization device such as an air or liquid sanitization device. Persons skilled in the art will appreciate that different wavelengths of light (e.g., different wavelengths of UV-C, UV-B, and/or UV-C, and/or sub 100 nm and or wavelengths greater than UV-A) may be provided about a tube providing a working area to insert light of that wavelength into the working area. Different wavelengths of light may, for example, provide improved different treatments for different types of contaminants. For example, one type of UV treatment may be utilized to optimize inactivation of virus using a photonic effect targeting the uracil of a virus while another type of UV treatment may be utilized to optimize impact of contaminants using a photonic effect targeting the thymine of a contaminant. Heat conductive materials such as heat sinks and heat sink fins may be attached to structures and each other using, for example, heat transfer pads (e.g., thermal pads) and/or heat transfer substances (e.g., thermal paste).

A device may be provided that may include fan blade operated by a motor that provides a working substance through the inlet of a working area so the substance can receive one or more types of treatments (e.g., a heat treatment and a UV-C treatment). Persons skilled in the art will appreciate that multiple types of treatments may be utilized. For example, heat may be introduced into a working area (e.g., by active heat generators or by heat sinks providing heat into a working channel) in order to impact a contaminant (e.g. inactivate a contaminant or render a contaminant inoperable). A tube may be provided to provide a treatment working area. A working area may be fabricated from one part or from multiple parts mechanically removably attached or permanently fixed (e.g., welded and/or adhered) together. An output may be provided so that air may flow out of a treatment working area. Persons skilled in the art will appreciate that materials forming an inlet and/or outlet may fabricated from different materials from a portion of a working area structure between an inlet and outlet. For example, the inlet and outlet portions may be non UV-C reflective on their surfaces facing a treatment working area such that UV-C does not reflect off those surfaces and out of the treatment working area. Furthermore, for example, any number of inlets and or outlets may be provided into a working area. For example, a working area may have one inlet and two or three or more outlets. As per another example, a working area may have one outlet and two or three or more inlets. As per another example, a working area may have two or three or more inlets and outlets and the number of inlets and outlets may be the same or may be different. Inlets and/or outlets may have different sizes and shapes and interior and exterior surface shapes and may be fabricated as one part or multiple parts form one or more of the same or different materials using one or more of the same or different processes. A substance may flow out through an output and may, for example exit a device and enter the environment of the device (e.g., in a ventilator setting may exit a UV-C sanitization device and enter a ventilator tube) or may enter a room (e.g., an elevator, hotel room, cruise ship room) with sanitized air. As per another example, treated air may be flow out of an outlet through a channel and may leave the device or chamber through one or more apertures in the structure providing that channel. After treated air leaves a portion of the device, the air may exit the device or may be flowed into another chamber. Persons skilled in the art will appreciate that UV-C LEDs may be mounted to the exterior of a tube providing a working area as well as one or more heat sinks and air may be flowed across the exterior of the tube providing the working channel (e.g., over surfaces of the heat sinks across the tube providing the working channel) and out through an outlet channel. In doing so, for example, treated air may be utilized to also remove heat from the device. In doing so, for example, air is not circulated from device 830 that is not treated. In circulating untreated air, a device may introduce more contaminants into a portion of an environment by more quickly spreading contaminated air. Additionally, certain contaminants may be impacted by heat. Accordingly, the removal of heat may provide, for example, a second type of treatment in order to increase the inactivation of contaminants and/or render more contaminants inoperable.

Persons skilled in the art will appreciate that an access door may be provided on one or more areas of an UV-C generating device and may be, for example, aligned with an outlet or an aperture of a tube providing a working area so that the access door may be opened and a cleaning brush may be utilized to clean the interior of the working channel. A lock may be provided on the access door and a keyhole may be provided on the lock so a key may be utilized to open the lock. Other security mechanisms can be provided such as, for example, a keypad entry that utilizes an entry code or a biometric access lock (e.g., fingerprint and/or retinal). Persons skilled in the art will appreciate that a UV-C generating device may be able to detect the status of an access door (e.g., whether the access door is opened or closed) and the device may restrict the UV-C light sources from turning on until circuitry confirms the access door is closed. Any number of access doors may be provided such as, for example, an access door about an inlet to receive a particulate filter which could also, for example, be utilized to receive a cleaning utensil (e.g., brush) and the cleaning utensil may be able to attach to and be removed from a structure located on the device. A chain or rope or other flexible structure may be utilized to keep the cleaning utensil secured to device 830 even when the cleaning utensil is removed from an attachment structure to the device and is being utilized by a user. Additionally, for example, a movable (e.g., pivotable) air direction fin (or fins) may be provided at inlet 834 so that, for example, air may be pointed to different areas of a working area. Doing so may, for example, increasing the impact of a cleaning protocol such that a cleaning protocol that increases airflow into a working are to clean the working area may be moved to provide air at different locations in order to improve the impact of the cleaning process.

A UV-C inactivation device may include any number of UV-C generating devices (e.g., three, four, more than four). Each UV-C generating device may be utilized, for example, to sterilize air and may include one or more fans (e.g., two fans each with two counter-rotating blades) to bring air into a working channel of the UV-C generating devices. A structure may be utilized to fix the UV-C generating devices together and may be utilized for example in a passageway such as an air duct.

Device 850 may be provided and may include flexible circuit board 894 that may include any number of light sources 851-855, any number of power circuitry 861, any number of mounting apertures 871, and any number of additional electronics 881. Flexible circuit board 894 may be wrapped into a shape to form a working area and/or wrapped around a structure (e.g., a tube with apertures) to form an inactivation area. Light sources may be provided about apertures that are filled with UV-C transparent material (e.g., quartz) such that light from UV-C light sources may be provided into a working area through the UV-C transparent material. The interior of a working area may be UV-C reflective such that, for example, UV-C in the working area is increased. Mounting apertures may be utilized to mount circuit board 894 to one or more structures (e.g., one or more heat sinks).

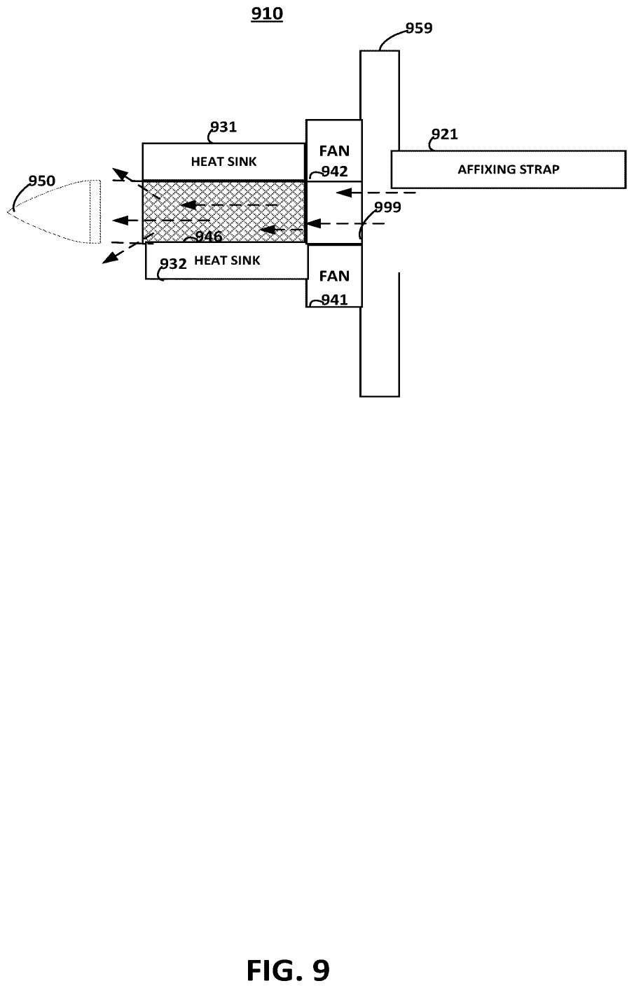

FIG. 9 shows device 910 that may include affixing strap 921 so that device 910 may be affixed, for example, to a user's face to provide a face mask. Air may be breathed through working area 946 and exhaled air may flow over cone 950. Heat sinks 931 and 932 may remove heat from UV-C sources that provide UV-C light in working area 946 and fans 941 and 942 may remove heat from the heat sinks (e.g., remove heat from heat sinks and/or heat sink fins). Fans 941 and 942 may also provide breathing assistance by moving air into a user as a user inhales and moving air out of mask 910 as a user exhales through, for example, controlled air channels that may open/close/divert air as a user breathes. In doing so, more filtering with air resistance may be added to potentially, for example, add certain types of filtration or protective devices for certain missions without increasing the air resistance perceived by a user inhaling and exhaling as would have occurred if fan powered assisted breathing was not provided. Structure 999 may permit, for example, air to flow through it and may include the ability to have sensors added to structure 999 such as, for example, airflow sensors, UV-C light detection sensors, humidity sensors, temperature sensors, and/or any other type of sensor for use, for example, by a device such as device 910.

Persons skilled in the art will appreciate that any types of fans may be provided on an air or virus or liquid inactivation device such as centrifugal and/or axial fans.

Persons skilled in the art will appreciate that UV-C LEDs may, for example be between 250 and 290 nm or, more particularly, between 260 and 280 nm or, more particularly, between 260 and 270 nm, or more particularly, between 260 and 265 nm, or more particularly be approximately 262 nm. Person skilled in the art will appreciate that each UV-C LED may, for example, provide UV-C light at an energy of at least 20 milliwatts or more or, more particularly, at an energy of at least 50 milliwatts or more, or, more particularly, at an energy of at least 70 milliwatts or more.

Persons skilled in the art will appreciate that UV-C air sanitation devices may be used for any UV-C sterilization purpose such as UV-C inactivation of viruses to create vaccines, and/or sanitize liquids, etc.).

Persons skilled in the art will appreciate that UV-C transparent materials may have at least 80 percent, 90 percent, 92 percent, or more than 92 percent UV-C transparency. UV-C LEDs may provide, for example, light between 220 and 280 nanometers (e.g., between 255 and 275 nanometers). A device may have, for example, at least 10, at least 20, and at least 30 UV-C LEDs.

Persons skilled in the art will appreciate that a UV-C LED may produce visible spectrum light and that one or more visible light sensors may be utilized to detect this light in order to, for example, detect the amount of UV-Cina working area to determine, for example, if a cleaning process should be initiated. Each UV-C LED may be operated independently and the amount of visible spectrum light compared to stored information associated with a clean state (e.g., a state when the device was manufactured or initially tested). In doing so, for example, the cleanliness of UV-C transparent material for a particular UV-C LED may be determined. Accordingly, a tube that provides a working area may have recessed portions and apertures associated to visible light sensors (and/or other sensor) and such sensors may be located at, for example, about each inlet/outlet of a device. Such sensors may be tilted to face into a working channel such that more light is received. In addition, or instead of, testing each light source independently (e.g., each UV-C light source independently) the UV-C LEDs may be tested in groups and may be tested multiple times. All the UV-C LEDs may also be turned ON and light sensed to determine a cleanliness profile for the device. In sensing multiple different UV-C LEDs operating at different times, a cleanliness profile may be determined for each UV-C transparent material that is associated with each LED as well as the cleanliness of different areas of UV-C reflective materials (or other materials) that may be provided on an inner surface of a working area. Persons skilled in the art will appreciate that visual indicators (e.g., light sources and/or displays) may be utilized to provide feedback on cleanliness and the cleanliness of different portions of a device as well as estimated sterilization impact at different operating modes. Furthermore, manual inputs may be provided so a user can perform a cleaning profile diagnostic so that after a cleaning a user can confirm the level of cleanliness that exist sin the device. Persons skilled in the art will appreciate that a cleanliness profile diagnostic may also, for example, be utilized to indicate if a UV light source is estimated to not be operational or operational at a particular diminished capacity. The operation of a device may be changed (e.g., autonomously) based on sensed data such as, for example, additional UV light sources may be activated and/or the intensity of particular UV-C sources may be increased.

Persons skilled in the art will appreciate that a light source calibration process may be utilized to calibrate devices. Efficacy levels may be adjusted based, in part, on calibration data. Integrating sphere(s) may be utilized as part of a process to calibrate UV-C LEDs based on determined output and controlled thresholds. Persons skilled in the art will appreciate that fiber optics may be utilized to transport UV light, such as UV-C light, to a working area, such as a tube. Multiple UV-C LEDs may be optically connected to UV-C fiber optics and these fiber optics may be combined through a UV-C fiber optic combiner and the combined UV-C fiber optic may be provided through, for example, an aperture of a structure (e.g., a tube) having a working area for receiving UV-C. UV-C optical modifiers may be provided at the end of a combined UV-C fiber optic to provide different UV-C output profiles.

Persons skilled in the art will appreciate that elements of any device herein may be utilized in any device herein. Persons skilled in the art will also appreciate that the present invention is not limited to only the embodiments described. Instead, the present invention more generally involves UV-C focus, amplification, and control. Persons skilled in the art will also appreciate that the apparatus of the present invention may be implemented in other ways then those described herein. All such modifications are within the scope of the present invention, which is limited only by the claims that follow.

What is claimed is:

1. A device comprising:
a first facemask housing attachable to and removable from a second facemask housing, wherein said first facemask housing includes:
a fan;
a plurality of ultraviolet type-C light emitting diodes located around a working area that receives external air, wherein at least two of said plurality of ultraviolet type-C light emitting diodes are centered at a wavelength between 250 and 275 nanometers, and said ultraviolet type-C light emitting diodes are operable to provide ultraviolet light to said working area;
a breathing channel in which said external air is moved from said working channel to the breathing proximity of a user after traveling through said working area.

2. The device of claim 1, wherein said second mask includes at least one mechanical filter.

3. The device of claim 1, further comprising a second fan.

4. The device of claim 1, wherein said working area is defined, at least in part, by a cylinder having a UV-C reflective interior.

5. The device of claim 1, wherein said working area is defined, at least in part, by a cylinder having a UV-C reflective interior, wherein a first UV-C reflective object is placed about an entrance of said cylinder to reflect UV-C light back into said cylinder, wherein said external air is operable to move around said first UV-C reflective object.

6. The device of claim 1, wherein said working area is defined, at least in part, by a cylinder having a UV-C reflective interior, wherein a first UV-C reflective object is placed about an entrance of said cylinder to reflect UV-C light back into said cylinder, a second UV-C reflective object is placed about an exit of said cylinder to reflect UV-C light back into said cylinder, wherein said external air is operable to move around said first UV-C reflective object and said second UV-C object.

7. The device of claim 1, wherein said second face mask includes at least two mechanical filters.

8. The device of claim 1, wherein said second face mask includes at least one air filter port operable for receiving an air filter.

9. The device of claim 1, wherein said second face mask includes at least two air filter ports operable each to receive an air filter.

10. The device of claim 1, wherein said first face mask includes a rechargeable battery.

11. The device of claim 1, wherein said first face mask includes a flow sensor for determining the direction and magnitude of air flowing through said working channel.

12. The device of claim 1, wherein said first face mask includes a UV-C sensor for sensing an amount of UV-C being generated in said working area.

13. The device of claim 1, wherein said first face mask includes a first UV-C sensor for sensing a first amount of UV-C being generated in said working area and a second UV-C sensor for sensing a second amount of UV-C being generated in said working area.
